(19) United States
(12) Patent Application Publication
(10) Pub. No.: US 2024/0368241 A1
(43) Pub. Date: Nov. 7, 2024

(54) SYNDECAN PEPTIDES

(71) Applicant: Queen Mary University of London, London (GB)

(72) Inventors: James Whiteford, London (GB); Giulia De Rossi, London (GB); Samantha Arokiasamy, London (GB)

(73) Assignee: Queen Mary University of London, London (GB)

(21) Appl. No.: 18/292,770

(22) PCT Filed: Jul. 25, 2022
(86) PCT No.: PCT/GB2022/051943

§ 371 Date: Jan. 26, 2024

(30) Foreign Application Priority Data
Jul. 26, 2021 (GB) 2110693.5

(51) Int. Cl. C07K 14/705; A61K 38/00; A61K 45/06; A61P 27/02

(52) U.S. Cl. CPC C07K 14/705; A61K 45/06; A61P 27/02; A61K 38/00

(57) ABSTRACT

The present invention relates to peptides and their use in treating diseases associated with angiogenesis. The present invention also relates to the use of peptides in treating diseases associated with vascular permeability. The peptides are based on syndecan (SDC) peptides.

### FIELD OF THE INVENTION

The present invention relates to peptides and their use in treating diseases associated with angiogenesis. The present invention also relates to the use of peptides in treating diseases associated with vascular permeability.

### BACKGROUND TO THE INVENTION

### Angiogenesis

Angiogenesis is a physiological process involving activation of endothelial cells from a quiescent state to a migratory and proliferative phenotype in response to specific biological signals to form new blood vessels. It is an essential feature of growth and development, heart and kidney function and wound healing. Pathological angiogenesis is involved in a number of diseases such as cancer and inflammatory conditions such as rheumatoid arthritis and atherosclerosis. It plays a critical role in the growth and spread of cancer and is therefore a key target in cancer therapy.

New blood vessel formation entails proliferation of endothelial cells and remodelling of the extracellular matrix (ECM). Integrins, which play a major role in this response, exist in various activation states on the cell surface and modulate the migratory and adhesive characteristics of cells through interactions with the ECM. Other cell surface receptors also interact with ECM ligands leading to signalling cascades that can alter the activation state of integrins. Syndecans are an example of such molecules.

A number of inhibitors have been developed to suppress angiogenesis. For example, small molecules such as sorafenib and pazopanib inhibit kinases that promote angiogenesis; bevacizumab targets vascular endothelial growth factor (VEGF), a potent pro-angiogenic signalling molecule. There are however a number of serious side effects commonly associated with the use of anti-angiogenic compounds for example haemorrhage, hypertension, lymphopenia and diarrhea. In addition in the case of VEGF targeting therapies there is also a significant number of patient non-responders (˜45%).

Thus there is a need for alternative therapies and methods for treating diseases associated with angiogenesis.

### Vascular Permeability

Aberrant vascular permeability involves the disassociation of junctions between endothelial cells (ECs), leading to leakage of fluids and bioactive molecules from blood vessels. Vascular permeability is a critical process in numerous disease pathologies typically involving oedema and/or inflammation including for example cancer and the early phases of diabetic retinopathy, macular degeneration.

In ocular diseases, vascular permeability can lead to swelling of the retina as well as reduction of oxygen supplying the retinal tissue, facilitating vision impairment. In diabetic retinopathy and macular degeneration, vessel leakage occurs early in the pathology of the disease, and is an exacerbating factor in the neovascularisation responses seen later in the pathology.

The main treatment for diseases associated with vascular permeability is anti-VEGF therapy, for example the use of VEGF-blocking antibodies or VEGFR kinase inhibitors. However, there is a high level of patients that are non-responsive or refractory to such treatment. A large number of anti-VEGF therapies also demonstrate a loss of efficacy over time and are associated with off target effects.

Thus there is a need for alternative therapies and methods for treating diseases associated with vascular permeability.

### Syndecans

Syndecans are a family of transmembrane receptors with roles in cell adhesion, migration and growth factor signalling. Each syndecan molecule comprises a short highly conserved cytoplasmic domain, a transmembrane domain and a larger extracellular domain (ectodomain). In mammals, there are four syndecan family members-syndecans-1,-2,-3 and-4.

In common with the other family members syndecan-2 and syndecan-3 have a short cytoplasmic domain, a single pass transmembrane domain and a larger extracellular domain which is substituted toward the N-terminus with heparan sulphate (HS) side chains and can be shed from the cell surface. Syndecan ectodomain shedding is a feature of many cell types and occurs in response to inflammatory stimuli. These shed moieties become soluble effectors of EC responses.

WO2016063042 describes syndecan-2 derived peptides as anti-angiogenic agents.

The present inventors have surprisingly found that peptides based on a portion of the syndecan-3 molecule have an unexpected anti-angiogenic effect. Furthermore, the inventors have surprisingly found that peptides based on a portion of the syndecan-2 molecule haves an unexpected effect on vascular permeability.

### SUMMARY OF THE INVENTION

The present inventors have surprisingly found that peptides based on a portion of the syndecan-3 molecule have an unexpected anti-angiogenic effect. Furthermore, the inventors have surprisingly found that peptides based on a portion of the syndecan-2 molecule have an unexpected effect on vascular permeability. Such peptides may be used alone or in combination for treatment of a variety of diseases.

The present invention thud provides peptides with anti-angiogenic activity and the ability to block vascular permeability, and nucleic acids that encode these peptides. The invention also provides methods, pharmaceutical compositions, and kits for treating diseases associated with angiogenesis and vascular permeability.

Accordingly, in a first aspect, the invention provides a peptide comprising an amino acid sequence having at least 70% identity to SEQ ID NO: 1. The peptide may comprise the amino acid sequence of SEQ ID NO: 2 or 7. The peptide may be up to 50 amino acids in length.

In a further aspect, the invention provides a fusion polypeptide comprising the peptide of the invention fused to a heterologous peptide. The heterologous peptide may comprise an amino acid sequence having at least 70% identity to SEQ ID NO: 3 or SEQ ID NO: 4, optionally wherein the fusion peptide comprises the amino acid sequence of SEQ ID NO: 19 or SEQ ID NO: 20. The peptide of the invention may be fused to the heterologous peptide using a linker, optionally wherein the linker is a peptide linker. The heterologous peptide may comprise an amino acid sequence having at least 70% identity to SEQ ID NO: 3 or SEQ ID NO: 4, optionally wherein the fusion peptide comprises the amino acid sequence of SEQ ID NO: 21 or SEQ ID NO: 22.

In a further aspect, the invention provides a combination of two peptides, the first peptide comprising an amino acid sequence having at least 70% identity to SEQ ID NO: 3 or SEQ ID NO: 4, the second peptide comprising an amino acid sequence having at least 70% identity to SEQ ID NO: 1. The first peptide may consist of up to 25 amino acids and include an amino acid sequence having at least 70% identity to SEQ ID NO: 3 or SEQ ID NO: 4, optionally wherein the first peptide comprises or consists of an amino acid sequence having at least 70% identity to up to 25 consecutive amino acid residues selected from: amino acid residues 120-144 of SEQ ID NO: 5 or amino acid residues 121-145 of SEQ ID NO: 6. The first peptide may consist of an amino acid sequence having at least 70% identity to SEQ ID NO: 3 or SEQ ID NO: 4.

In a further aspect, the invention provides a nucleic acid construct encoding a peptide, fusion polypeptide, or combination of peptides of the invention.

In a further aspect, the invention provides a vector comprising a nucleic acid construct of the invention.

In a further aspect, the invention provides a cell comprising a nucleic acid construct or a vector of the invention.

In a further aspect, the invention provides a pharmaceutical composition comprising a pharmaceutically acceptable carrier and a peptide, fusion polypeptide, combination of peptides, or nucleic acid construct of the invention.

In a further aspect, the invention provides a peptide, fusion polypeptide, combination of peptides, nucleic acid construct or pharmaceutical composition for use in a method of therapy practised on the human or animal body.

In a further aspect, the invention provides a peptide, fusion polypeptide, combination of peptides, nucleic acid construct or pharmaceutical composition for use in a method of treatment of a disease associated with angiogenesis. The disease may be cancer, arthritis, psoriasis, asthma, atherosclerosis or an ocular disease selected from the group consisting of diabetic retinopathy, exudative (wet) or nonexudative (dry) macular degeneration (AMD), corneal graft rejection, corneal neovascularisation, retinopathy of prematurity (ROP), retinal artery or vein occlusion, neovascular glaucoma and sickle cell retinopathy.

In a further aspect, the invention provides a method for the treatment of a disease associated with angiogenesis comprising administering to a subject in need thereof a therapeutically effective amount of a peptide, fusion polypeptide, combination of peptides, nucleic acid construct or pharmaceutical composition of the invention.

In a further aspect, the invention provides a kit comprising a peptide, fusion polypeptide, combination of peptides, nucleic acid construct or pharmaceutical composition of the invention.

In a further aspect, the invention provides a peptide for use in a method of treating a patient with a disease associated with vascular leakage, wherein the peptide comprises an amino acid sequence having at least 70% identity to SEQ ID NO: 3 or SEQ ID NO: 4, optionally wherein the peptide is as defined in any one of claims 8 to 10. The peptide may be administered as part of a combination according to the invention. The disease may not have progressed to neovascularisation.

In a further aspect, the invention provides a peptide for use in a method of preventing progression of a disease associated with vascular leakage to a disease comprising neovascularisation, optionally wherein the peptide comprises an amino acid sequence having at least 70% identity to SEQ ID NO: 3 or SEQ ID NO: 4.

The disease may be an ocular disease. The ocular disease may be early-stage AMD, diabetic retinopathy or diabetic macular oedema. The ocular disease may be associated with oedema and/or inflammation.

The disease may be selected from cancer, a solid tumour, rheumatoid arthritis, lymphoedema, asthma, ventilator induced-lung injury, acute lung injury, atherosclerosis, ischemic stroke, psoriasis, an inflammatory bowel disorder, and/or myocardial infarction, optionally wherein said cancer is ovarian cancer or lung cancer, preferably epithelial ovarian cancer or nonsquamous non-small cell lung cancer. The disease may be a solid tumour and the peptide may be administered in combination with a chemotherapeutic agent or irradiation treatment, optionally thoracic irradiation treatment.

The disease may be refractory to treatment with an anti-VEGF antagonist.

### BRIEF DESCRIPTION OF THE FIGURES

FIG. 1. The syndecan-3 extracellular core protein inhibits angiogenic sprout formation from rat aortic rings. Schematic diagram showing the GST fusion protein consisting of GST fused at the N-terminus of the complete coding sequence of the murine Syndecan-3 extracellular core protein. This protein was expressed and purified from bacteria and was tested in the rat aortic ring angiogenic sprouting assay. Aortas were dissected from rats before being sliced into 1 mm wide rings. They are then embedded in collagen I with 0.5 μM of either GST or S3ED purified fusion proteins. 10 ng/ml of VEGF-A was then added into feeding media to encourage angiogenesis. After 7 days, angiogenic sprouts were counted. Images were captured on day 7 using a Hamamatsu Orca ER digital camera and processed using Cell M software. Data is from at least 10 rings per condition from 8 mice. (A) Phase contrast micrographs of aortic rings after 7 days. (B) Graph showing the number of sprouts formed after 7 days (*** p≤0.005).

FIG. 2. Miniaturization strategy to identify minimum peptide sequence for inhibition of angiogenesis. Schematic diagram showing the series of deletion mutants generated to ascertain the anti-angiogenic peptide sequence contained in syndecan-3 (A). Purified GST fusion proteins were used at a concentration of 0.5 μM in scratch wound migration assays using confluent monolayers of HUEVCs. Wound closure was measured after 7 hours. In this way we identified that the antiangiogenic properties of Syndecan-3 reside between P195 and A221 of the protein.

FIG. 3. Peptides corresponding to the anti-angiogenic regions of Human and Murine syndecan-3 inhibit HUVEC cell migration and have comparable efficacy to QM107. Comparison of the antiangiogenic sequences in the murine and human forms of syndecan-3 (A). Amino acids which are different are highlighted in yellow. Scratch wound cell migration assays comparing the anti-migratory effects of QM107, QM111 and QM111M on HUVECS (B). Of note greater inhibition is achieved when QM107 and QM111 are used in combination (n=5/condition, *** p≤0.001, **** p≤0.0001). Representative micrographs showing extent of inhibition of

HUVEC cell migration when treated with QM111.

FIG. 4. Peptides corresponding to the anti-angiogenic regions of Human and Murine syndecan-3 inhibit angiogenic sprout formation and have comparable efficacy to QM107. Comparison of the antiangiogenic sequences in the murine and human forms of syndecan-3 (A). Amino acids which are different are highlighted in yellow. Rat aortic rings where treated with 0.5 μM of either QM107, QM111, QM111M or in combination. Angiogenic sprout formation is inhibited to a similar degree when treated either QM107. QM111 or QM111M. A combination of QM107 and QM111 appears to be most efficacious (n=30-31 rings/condition. **** p≤0.0001).

FIG. 5. QM111 blocks pathological neovascularization in a model of Diabetic retinopathy. Data from an oxygen-induced model of retinopathy study. In this study. OIR was generated by exposing mice pups to 75% oxygen for 5 days from P7-P12. On P13, mice are injected with PBS (left eye) and 0.5 μM QM111 (right eye). They were then placed into 20% oxygen from P13-P17 where they underwent hypoxia. which led to neovascularisation taking place. OIR samples were collected at P17 and retinal structures were stained with isolectin GS-IB4. Neovascularisation was quantified by measuring the total areas where preretinal tufts were present. The graph shows the percentage of neovascularisation in the control eye injected with PBS vs. the treated eye injected with QM111. Data is from 8 mice.

FIG. 6. Miniaturization Strategy based on comparison between Mouse and Human Sequences. Since both peptides corresponding to the antiangiogenic regions of both the human and mouse form of syndecan-3 had similar anti-angiogenic properties we compared the sequences in the hope of identifying the smallest active form of QM111. We synthesized the 9 amino acid sequence QM111T.

FIG. 7: QM111T inhibits choroidal neovascularization. Murine choroid explants were embedded in a Collagen I matrix and supplemented with 30 ng/ml VEGF and either 0.5 μM of QM111, QM111T, QM107 or a combination. QM111 and QM111T have similar a similar inhibitory affect on sprouting from Choroid explants (n=15-18 choroid explants/condition, *** p≤0.0001 **** p≤0.0001).

FIG. 8. Combination therapy using QM107 and QM111 results in less variability in angiogenesis assays. Comparison of the standard deviation of scratch wound EC migration assays in (A), angiogenic sprout formation form aortic rings (B) and sprout formation from murine choroid membrane explants (C). The red line highlights the point at which the lowest standard deviation is observed.

FIG. 9. QM107 blocks vascular permeability responses to VEGFA and Bradykinin. 6-8 Week old mice were anesthetized by i.m. injection of 1 ml/kg ketamine (40 mg) and xylazine (2 mg) in saline solution. The back skin was shaved using an electric razor. Mice then received Evans Blue dye (0.5% in PBS, 5 μl per g bodyweight) i.v. through the tail vein. Afterwards, 50 μl of PBS containing either 100 ng of VEGFA or 100 μg of Bradykinin or PBS alone with or without QM107 dose were injected s.c. in the mouse dorsal skin. After 90 min animals were sacrificed by cervical dislocation. Dorsal skin was removed and injected sites were cut out as circular patches using a metal puncher (˜8 mm in diameter). Samples were then incubated in 250 μl of formamide at 56° C. for 24 h to extract Evans Blue dye from the tissues. The amount of accumulated Evans Blue dye was quantified by spectroscopy at 620 nm using a Spectra MR spectrometer (Dynex technologies Ltd., West Sussex, UK). Results are presented as the optical density at 620 nm (OD620) per mg tissue and per mouse in (A) and representative images are shown in (B). (n=9 animals/condition) *p<0.05. ** p<0.01.

### BRIEF DESCRIPTION OF SEQUENCES

SEQ ID NO: 1 is an active amino acid sequence fragment of human Syndecan-3 (SDC3).

SEQ ID NO: 2 is a longer active amino acid sequence of human SDC3.

SEQ ID NO: 3 is the amino acid sequence of residues 123-140 of human Syndecan-2 (SDC2).

SEQ ID NO: 4 is the amino acid sequence of residues 124-141 of mouse SDC2.

SEQ ID NO: 5 is the amino acid sequence of human SDC2.

SEQ ID NO: 6 is the amino acid sequence of mouse SDC2.

SEQ ID NO: 7 is an active amino acid sequence fragment of mouse SDC3.

SEQ ID NO: 8 is the amino acid sequence of human SDC3.

SEQ ID NO: 9 is the amino acid sequence of mouse SDC3.

SEQ ID NO: 10 is a nucleotide sequence encoding SEQ ID NO:1.

SEQ ID NO: 11 is the nucleotide sequence of the longer active amino acid sequence of human SDC3.

SEQ ID NO: 12 is the nucleotide sequence for residues 123-140 of human SDC2.

SEQ ID NO: 13 is the nucleotide sequence for residues 124-141 of mouse SDC2.

SEQ ID NO: 14 is the nucleotide sequence of human SDC2.

SEQ ID NO: 15 is the nucleotide sequence of mouse SDC2.

SEQ ID NO: 16 is a nucleotide sequence encoding SEQ ID NO: 7.

SEQ ID NO: 17 is the nucleotide sequence of human SDC3.

SEQ ID NO: 18 is the nucleotide sequence of mouse SDC3.

SEQ ID NO: 19 is the amino acid sequence of the fusion of the minimal active amino acid sequence of human Syndecan-3 (SDC3) and amino acid sequence of residues 123-140 of human Syndecan-2 (SDC2). Residues 123-140 of human Syndecan-2 (SDC2) are bound to the C-terminus of the minimal active amino acid sequence of human Syndecan-3 (SDC3).

SEQ ID NO: 20 is the amino acid sequence of the fusion of the minimal active amino acid sequence of human Syndecan-3 (SDC3) and amino acid sequence of residues 123-140 of human Syndecan-2 (SDC2). Residues 123-140 of human Syndecan-2 (SDC2) are bound to the N-terminus of the minimal active amino acid sequence of human Syndecan-3 (SDC3).

SEQ ID NO: 21 is the amino acid sequence of the fusion of the minimal active amino acid sequence of human Syndecan-3 (SDC3) and amino acid sequence of residues 123-140 of human Syndecan-2 (SDC2) via a peptide linker. Residues 123-140 of human Syndecan-2 (SDC2) are bound to the C-terminus of the minimal active amino acid sequence of human Syndecan-3 (SDC3) via the peptide linker.

SEQ ID NO: 22 is the amino acid sequence of the fusion of the minimal active amino acid sequence of human Syndecan-3 (SDC3) and amino acid sequence of residues 123-140 of human Syndecan-2 (SDC2) via a peptide linker. Residues 123-140 of human Syndecan-2 (SDC2) are bound to the N-terminus of the minimal active amino acid sequence of human Syndecan-3 (SDC3) via the peptide linker.

### DETAILED DESCRIPTION

### Definitions

“Angiogenesis” refers to the process of formation of new blood vessels. Angiogenesis requires the collective action of numerous pro-and anti-angiogenic factors to provide the signals necessary for the activation of endothelial cells to form new blood vessels. The angiogenic process involves a number of steps including enzymatic degradation of capillary basement membrane, endothelial cell (EC) proliferation and migration, invasion through the extracellular matrix and tubulogenesis.

“Tubulogenesis”, or “tubule formation”, as used herein refers to the development of endothelial cell tubes with an inner lumen generated by a polarised movement of the cells in response to pro-angiogenic signals.

“Anti-angiogenic activity” refers to suppression or inhibition of angiogenesis. “Anti-angiogenic peptides” are peptides that have anti-angiogenic activity.

A “peptide” refers to a chain of amino acid residues linked by peptide bonds. The terms “peptide” and “polypeptide” are used interchangeably.

Throughout this specification, amino acids may be referred to using the three letter and one letter codes as follows: glycine (G or Gly), alanine (A or Ala), valine (V or Val), leucine (L or Leu), isoleucine (I or lie), proline (P or Pro), phenylalanine (F or Phe), tyrosine (Y or Tyr), tryptophan (W or Trp), lysine (K or Lys), arginine (R or Arg), histidine (H or His), aspartic acid (D or Asp), glutamic acid (E or Glu), asparagine (N or Asn), glutamine (Q or Gin), cysteine (C or Cys), methionine (M or Met), serine (S or Ser) and Threonine (T or Thr). Where a residue may be aspartic acid or asparagine, the symbols Asx or B may be used. Where a residue may be glutamic acid or glutamine, the symbols Glx or Z may be used.

“Identity” as known in the art is the relationship between two or more polypeptide sequences or two or more polynucleotide sequences, as determined by comparing the sequences. Typically, identity of a given sequence to a specified sequence is measured over the whole length of the specified sequence, or may be measured over a specified contiguous part thereof. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic acids Research, 12, 387 (1984), BLASTP, BLASTN, and FASTA (Atschul et al., J. Molec. Biol. 215, 403 (1990).

The PILEUP and BLAST algorithms can be used to calculate identity or line up sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These 10 initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, 20 N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P (N)), which provides an indication of the probability by which a match 25 between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

“Vascular permeability” refers to the permeability of blood vessels that allows for the flow of fluids, molecules and cells in and out of the vessels. “Vascular permeability” and “vascular leakage” are used interchangeably.

### Peptides

In a first aspect, the invention provides a peptide comprising, consisting essentially of, or consisting of an amino acid sequence having at least 70% identity to SEQ ID NO: 1.

The peptide is typically at least 9 amino acid residues in length. The peptide may be at least 10, 15, 20, or 30 amino acids in length. The peptide may be no more than 20, 25, 30, 40, 50, 60, 70, 80, 90 or 100 amino acid residues in length. The peptide may be between 9 to 15, 9 to 20, 9 to 30, 9 to 40, 9 to 50, 9 to 60, 9 to 70, 9 to 80, 9 to 90 or 9 to 100 amino acid residues in length,

The peptide may be 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues in length.

The peptide of any of the above lengths typically comprises an amino acid sequence having at least 70% identity to SEQ ID NO: 1. The peptide of any of the above lengths may comprise an amino acid sequence having at least 75%, 80%, 85%, 90%, 95% or 100% identity to SEQ ID NO: 1. The peptide of any of the above lengths may comprise an amino acid sequence having at least 1, 2 or 3 substitutions, typically conservative amino acid substitutions with respect to SEQ ID NO: 1 and/or having 1 or 2 deletions with respect to SEQ ID NO: 1, typically at the N- and/or C-terminus of SEQ ID NO: 1. The peptide may comprise an amino acid sequence having 1 or 2 insertions with respect to SEQ ID NO:1.

A conservative change, which replaces the amino acid with another amino acid of similar chemical structure, similar chemical properties or similar side-chain volume. The amino acids introduced may have similar polarity, hydrophilicity or hydrophobicity to the amino acids they replace. Conservative amino acid changes are well known in the art and may be selected in accordance with the changes defined in Table A. Where amino acids have similar polarity, this can also be determined by reference to the hydropathy scale for amino acid side chains (Table B). Conservative amino acid changes may also be determined by reference to the Point Accepted Mutation (PAM) or BLOcks SUbstitution Matrix (BLOSUM) family of scoring matrices for conservation of amino acid sequence. Thus, conservative amino acid changes may be members of an equivalence group, being a set of amino acids having mutually positive scores in the similarity representation of the scoring matrix selected for use in an alignment of the reference and mutant polypeptide chains.

TABLE A
Physical characteristics of amino acids

| | | |
|---|---|---|
| NON-AROMATIC | Non-polar | G A P |
| | | I L V |
| | Polar - uncharged | C S T M |
| | | N Q |
| | Polar - charged | D E |
| | | H K R |
| AROMATIC | | H F W Y |

TABLE B
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Thus, for example, valine at position 8 of SEQ ID NO: 1 can be substituted for another non-polar amino acid, for example glycine, alanine, proline, isoleucine or leucine. As shown herein, in the corresponding mouse sequence, SEQ ID NO: 7, position 8 is occupied by alanine, illustrating the possibility of incorporating conservative substitutions whilst maintaining activity of the peptide, such as anti-angiogenic activity.

The peptide may comprise an amino acid sequence having at least 70%, 75%, 80%, 85%, 90%, 95% or 100% identity to SEQ ID NO: 2 or 7. The peptide may have any of the lengths mentioned above.

The peptide of SEQ ID NO: 1 has anti-angiogenic activity, as illustrated in the Examples. Thus, the above peptides comprising, consisting essentially of, or consisting of an amino acid sequence having at least 70% identity to SEQ ID NO: 1 are typically anti-angiogenic peptides. The peptide of SEQ ID NOs: 2 and 7 also have anti-angiogenic activity. Thus the above peptides comprising, consisting essentially of, or consisting of an amino acid sequence having at least 70% or greater identity to SEQ ID NOs: 2 or 7 are typically anti-angiogenic peptides

Anti-angiogenic activity may be determined by any suitable assay in the art. A suitable assay may be, for example, an angiogenic sprout assay. In such assays, angiogenesis in a sample tissue is induced by using factors such as VEGF-A, and the number of angiogenic sprouts are counted. The assay can be conducted in the presence and absence of the expression of the peptide of interest (e.g. the peptide of the invention), and the number of angiogenic sprouts can be counted in order to evaluate the anti-angiogenic activity of the peptide (see Examples 2, 4 and 5, for example).

The anti-angiogenic activity of a variant peptide based on SEQ ID NOs: 1, 7 or 8 will typically have at least 50%, 60%, 70%, 80%, 90% or 100% activity compared to SEQ ID NOs: 1, 7 or 8 respectively.

### Fusion Polypeptides

The invention also provides a fusion polypeptide comprising any of the peptides or amino acid sequences of the invention described above fused to a heterologous peptide, such as an amino acid sequence having at least 70% identity to SEQ ID NO: 1 fused to a heterologous peptide.

A “heterologous peptide” refers to a peptide having an amino acid sequence that is of different origin to a peptide of the invention. For example, the heterologous peptide may have an amino acid sequence that is not comprised in a peptide of the invention, or not comprised in full-length SDC3 of SEQ ID NO: 8 or 9. The heterologous peptide may impart desired characteristics to the anti-angiogenic peptide for example increased stability, enhanced transport or simplified purification or detection.

In the fusion polypeptide, the heterologous peptide may be fused to the N-or C-terminus of the peptides or amino acid sequences of the invention.

In an embodiment, an active amino acid sequence fragment of Syndecan-3 (SDC3) is fused to a heterologous peptide based on residues 123-140 of human Syndecan-2 (SDC2) or residues 124 to 141 of mouse Syndecan-2 (SDC2).

In an embodiment, an amino acid sequence having at least 70% identity to SEQ ID NO: 1 is fused to an amino acid sequence having at least 70% identity to SEQ ID NO: 3. An amino acid sequence having a sequence of SEQ ID NO: 1 may be fused to an amino acid sequence having a sequence of SEQ ID NO: 3, as shown in SEQ ID NO: 19 and SEQ ID NO: 20. Preferably the amino acid sequence having at least 70% identity to SEQ ID NO: 3 or having the sequence of SEQ ID NO: 3 is fused to the C-terminus of the amino acid sequence having at least 70% identity to SEQ ID NO: 1 or having a sequence of SEQ ID NO: 1 respectively, as shown for example in SEQ ID NO: 19.

In an embodiment, an amino acid sequence having at least 70% identity to SEQ ID NO: 1 is fused to an amino acid sequence having at least 70% identity to SEQ ID NO: 4. An amino acid sequence having a sequence of SEQ ID NO: 1 may be fused to an amino acid sequence having a sequence of SEQ ID NO: 4.

To create the fusion polypeptide of the invention, the peptide of the invention is typically covalently linked to the heterologous peptide. The peptide of the invention is typically genetically fused to the heterologous peptide. The peptide of the invention is genetically fused to heterologous peptide if the whole construct is expressed from a single polynucleotide sequence. The coding sequences of the peptide of the invention and the heterologous peptide may be combined in any way to form a single polynucleotide sequence encoding the construct. They may be genetically fused in any configuration. They are typically fused via their terminal amino acids. For instance, the amino terminus of the peptide of the invention may be fused to the carboxy terminus of the heterologous peptide and vice versa.

The peptide of the invention may be attached directly to the heterologous peptide. The peptide of the invention is preferably attached to the heterologous peptide using one or more linkers. The one or more linkers may be designed to constrain the mobility of the peptides. Suitable linkers include, but are not limited to, chemical crosslinkers and peptide linkers. Peptide linker are preferred if the peptide of the invention and heterologous peptide are genetically fused. Preferred linkers are amino acid sequences (i.e. peptide linkers). The length, flexibility and hydrophilicity of the peptide linker are typically designed such that it does not to disturb the functions of the peptide of the invention. Preferred flexible peptide linkers are stretches of 2 to 20, such as 4, 6, 8, 10 or 16, serine and/or glycine amino acids. More preferred flexible linkers include (SG)1, (SG)2, (SG)3, (SG)4, (SG)5 and (SG)8 wherein S is serine and G is glycine. Preferred rigid linkers are stretches of 2 to 30, such as 4, 6, 8, 16 or 24, proline amino acids. More preferred rigid linkers include (P)12 wherein P is proline.

When the peptide of the invention is attached to the heterologous peptide using one or more linkers, the heterologous peptide may be fused to the N- or C-terminus of the peptide of the invention via the linker.

In an embodiment, an active amino acid sequence fragment of Syndecan-3 (SDC3) is fused to a heterologous peptide based on residues 123-140 of human Syndecan-2 (SDC2) or residues 124 to 141 of mouse Syndecan-2 (SDC2) using a linker. The linker may be at least 1, 2, 3, 4, 5 or more amino acids in length. The linker may consist of or comprise Gly-Gly-Ser.

In an embodiment, an amino acid sequence having at least 70% identity to SEQ ID NO: 1 is fused to an amino acid sequence having at least 70% to SEQ ID NO: 3 via a linker. An amino acid sequence having a sequence of SEQ ID NO: 1 may be fused to an amino acid sequence having a sequence of SEQ ID NO: 3 via a linker, as shown in SEQ ID NO: 21 and SEQ ID NO: 22. Preferably the amino acid sequence having at least 70% identity to SEQ ID NO: 3 or having a sequence of SEQ ID NO: 3 is fused via a linker to the C-terminus of the amino acid sequence having at least 70% identity to SEQ ID NO: 1 or having a sequence of SEQ ID NO: 1 respectively, as shown for example in SEQ ID NO: 21.

In an embodiment, an amino acid sequence having at least 70% identity to SEQ ID NO: 1 is fused to an amino acid sequence having at least 70% identity to SEQ ID NO: 4 via a linker. An amino acid sequence having a sequence of SEQ ID NO: 1 may be fused to an amino acid sequence having a sequence of SEQ ID NO: 4 via a linker.

The peptide of the invention may be transiently attached to the heterologous peptide by a hex-his tag or Ni-NTA. They may also be modified such that they transiently attach to each other. The peptide of the invention may also be attached to the heterologous peptide via cysteine linkage. This can be mediated by a bi-functional chemical linker or by a polypeptide linker with a terminal presented cysteine residue.

The heterologous peptide may be an epitope tag or purification tag or cell-surface display tag or a tag that enables or facilitates systemic peptide delivery or delivery and targeting to a specific organ or to a tumour, or facilitates transfer across a barrier such as skin or gut or blood brain barrier. Suitable tags are known in the art. Suitable tags include, but are not limited to, AviTag, calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, SpyTag, SnoopTag, BCCP (Biotin Carboxyl Carrier Protein), Glutathione-S-transferase-tag, Green fluorescent protein-tag, Halo-tag, Maltose binding protein-tag, Nus-tag, Thioredoxin-tag,Strep-tag, Skin permeating and cell entering (SPACE)-tag, TD1-tag, magainin tag, TAT-tag, penetratin-tag, cell penetrating peptide (CPP)-tag, fluorescence tag, Fc tag. Fluorescent tag polypeptides include but are not limited to green fluorescent protein, red fluorescent protein, yellow fluorescent protein, cyan fluorescent protein and their derivatives. The heterologous peptide may be a signal peptide, such as an IgK peptide.

The fusion polypeptide may be labelled with a detectable label. The detectable label may be any of those discussed above.

### Peptide Combinations

The invention also provides a combination of two peptides. In an embodiment, the first peptide may comprise an amino acid sequence having at least 70% identity to SEQ ID NO: 3 or SEQ ID NO: 4, the second peptide comprising an amino acid sequence having at least 70% identity to SEQ ID NO: 1.

In the combination of peptides of the invention, the second peptide may be as described in any of the embodiments above.

The first peptide may comprise, consist essentially of or consist of an amino acid sequence having at least 70% identity to SEQ ID NO: 3 or SEQ ID NO: 4. SEQ ID NO: 3 depicts the amino acid sequence of residues 123 to 140 of a human syndecan-2 molecule. SEQ ID NO: 4 depicts the amino acid sequence of residues 124 to 141 of a mouse syndecan-2 molecule.

In the combination of peptides of the invention, the first peptide is typically at least 15, 16, 17 or 18 amino acid residues in length. The peptide may be no more than 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 amino acid residues in length. The peptide may be between 18 to 20, 18 to 30, 18 to 40, 18 to 50, 18 to 60, 18 to 70, 18 to 80, 18 to 90 or 18 to 100 amino acid residues in length. The first peptide may consist of up to 25 amino acids and include an amino acid sequence having at least 70% identity to: (i) SEQ ID NO: 3 or SEQ ID NO: 4; or (ii) up to 25 consecutive amino acid residues selected from: amino acid residues 120-144 of SEQ ID NO: 5 or amino acid residues 121-145 of SEQ ID NO: 6.

The peptide of any of the above lengths may comprise an amino acid sequence having at least 1, 2, 3, 4 or 5 substitutions, typically conservative amino acid substitutions with respect to SEQ ID NOs: 3 or 4 and/or having 1, 2, 3 or 4 deletions with respect to SEQ ID NOs: 3 or 4, typically at the N- and/or C-terminus of SEQ ID NOs: 3 or 4. The peptide may comprise an amino acid sequence having 1, 2, 3 or 4 insertions with respect to SEQ ID NOs: 3 or 4.

The peptide of any of the above lengths may comprise an amino acid sequence having: (i) at least 1, 2, 3, 4, 5, 6 or 7 substitutions, typically conservative amino acid substitutions with respect to amino acid residues 120-144 of SEQ ID NO: 5 or amino acid residues 121-145 of SEQ ID NO: 6 and/or having 1, 2, 3, 4, 5 or 6 deletions with respect to amino acid residues 120-144 of SEQ ID NO: 5 or amino acid residues 121-145 of SEQ ID NO: 6, typically at the N- and/or C-terminus of amino acid residues 120-144 of SEQ ID NO: 5 or amino acid residues 121-145 of SEQ ID NO: 6. The peptide may comprise an amino acid sequence having 1, 2, 3, 4, 5 or 6 insertions with respect to amino acid residues 120-144 of SEQ ID NO: 5 or amino acid residues 121-145 of SEQ ID NO: 6.

The first peptide of any of the above lengths may comprise a sequence having at least 70% identity to SEQ ID NO: 3 or SEQ ID NO: 4, or to up to 25 consecutive amino acid residues selected from: amino acid residues 120-144 of SEQ ID NO: 5 or amino acid residues 121-145 of SEQ ID NO: 6. The first peptide of any of the above lengths may comprise a sequence having at least 75%, 80%, 85%, 90%, 95% or 100% identity to SEQ ID NO: 3 or SEQ ID NO: 4, or to up to 25 consecutive amino acid residues selected from: amino acid residues 120-144 of SEQ ID NO: 5 or amino acid residues 121-145 of SEQ ID NO: 6.

The first peptide of any of the above lengths may consist of up to 25 amino acids and include an amino acid sequence having at least 70% identity to SEQ ID NO: 3 or SEQ ID NO: 4.

The first peptide of any of the above lengths may comprise, consist essentially of or consist of an amino acid sequence having at least 70% identity to up to 25 consecutive amino acid residues selected from: amino acid residues 120-144 of SEQ ID NO: 5 or amino acid residues 121-145 of SEQ ID NO: 6.

The peptides of SEQ ID NOs: 3 to 6 have anti-angiogenic activity, as illustrated in the Examples. Thus, the above peptides comprising, consisting essentially of, or consisting of an amino acid sequence having at least 70% identity to: (i) SEQ ID NO: 3 or SEQ ID NO: 4; or (ii) up to 25 consecutive amino acid residues selected from: amino acid residues 120-144 of SEQ ID NO: 5 or amino acid residues 121-145 of SEQ ID NO: 6 are typically anti-angiogenic peptides. Anti-angiogenic activity may be determined by angiogenic sprouting assays, as described in the “Peptides” section above.

The peptides of SEQ ID NOs: 3 to 6 are also able to block vascular permeability. Therefore, the above peptides comprising, consisting essentially of, or consisting of an amino acid sequence having at least 70% identity to: (i) SEQ ID NO: 3 or SEQ ID NO: 4; or (ii) up to 25 consecutive amino acid residues selected from: amino acid residues 120-144 of SEQ ID NO: 5 or amino acid residues 121-145 of SEQ ID NO: 6 may also block or reduce vascular permeability.

Vascular permeability can be evaluated by suitable assays in the art, in in vivo, ex vivo and in vitro conditions. For example, Evans Blue dye can be used to measure vascular permeability. Evans Blue dye is a blue dye that binds to serum albumin. In healthy tissue, staining with the dye is restricted within blood vessels. In diseases associated with vascular permeability, the endothelium becomes permeable to albumin, and thus tissues will have increased blue colouring. The amount of dye accumulation can be measured by standard methods in the art, such as spectroscopy.

Alternative ways to evaluate vascular permeability include the use of dextran labelled with a component such as FITC. Dextran can bind to red blood cells, platelets and vascular endothelium. Therefore, in diseases associated with vascular permeability, more labelled dextran can be seen in tissues in a similar manner to Evans Blue dye. Labelled dextran can be imaged using standard methods in the art, such as microscopy.

Labelled fluorescent beads of different sizes can also be used. The differing sizes of the beads can assess the extent of damage caused by vascular permeability. If larger fluorescent beads can be seen within the endothelium, then it indicates larger permeability. The labelled fluorescent beads can be imaged using standard methods in the art, such as microscopy.

For in vitro studies, the conductance of endothelial monolayers can be evaluated as a measure of vascular permeability.

Blocking or reducing vascular permeability refers to preventing or reducing the amount of permeability in the endothelium, such as in of a tissue of interest displaying aberrant vascular permeability. The peptides may prevent, treat or reduce aberrant or abnormal vascular permeability. The peptides may prevent, treat or reduce oedema or swelling or may prevent or reduce one or more other symptoms associated with vascular permeability. The peptides may restore normal vascular permeability.

The peptides may restore sufficient vascular permeability to enhance the effect of a therapy. For example, if a peptide is administered to a patient with a solid tumour associated with aberrant vascular permeability, the peptide may block vascular permeability such that permeability of the tumour to an agent of interest, such as a chemotherapeutic agent, is increased. The blocking or reduction of vascular permeability may result in higher localisation and thus efficacy of an agent in a target area, for example a chemotherapeutic agent in a tumour.

The peptides may also block vascular permeability that has been caused by a side effect of a treatment. For example, thoracic irradiation treatment can treat tumours, but a side effect can be vascular permeability. Thus the administration of the peptides can facilitate the reduction or blocking of vascular permeability caused by side effects of treatments. As a result, the peptides can improve the overall effect of treatments where vascular permeability is a side effect.

In an embodiment of the first aspect of the invention the peptide or combination of peptides is isolated. “Isolated” refers to material removed from its original environment. The original environment could be a natural environment for example inside a cell. An isolated peptide or peptides as used herein refers to a peptide which is at least 20% pure, preferably at least 40% pure, more preferably at least 60% pure, even more preferably at least 80% pure, most preferably at least 90% pure, and even most preferably at least 95% pure, as determined by SDS-PAGE.

Peptides of the invention may be produced by recombinant means, for example by expression of a nucleic acid construct as disclosed herein in a suitable vector, or by solid phase synthesis.

It should be appreciated that amino acid substitutions or insertions to the sequences disclosed herein that are within the scope of the present invention can be made using naturally occurring or non-naturally occurring amino acids. For example, D-amino acids can be incorporated in the peptides of the invention, for example as substitution(s) for corresponding L-amino acids. This may improve resistance to proteolytic activity.

Peptides of the invention may be modified to improve their characteristics such as their half-life, for example by PEGylation.

### Nucleic Acids, Vectors, and Cells

In a second aspect the invention provides a nucleic acid construct encoding a peptide, a fusion polypeptide, or a combination of peptides according to the first aspect.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

In another embodiment of the invention, a nucleic acid sequence encoding a peptide of the invention may comprise, consist essentially of or consist of a nucleic acid sequence having at least 70% identity, at the nucleic acid level, to any of the nucleic acid sequences disclosed herein, for example any of the sequences depicted in SEQ ID NOs: 10 to 13 or 16 or any fragment thereof. More preferably, the nucleic acids may have at least 60%, 70%, 75%, 80%, 85%, 90%, 91%, 92%, 93%, 94% and still more preferably at least 95%, 96%, 97% or 98% (still more preferably at least 99%) identity, at the nucleic acid level, to any of the nucleic acid sequences disclosed herein, for example the sequences depicted in SEQ ID NOs: 10 to 13 or 16 or a fragment thereof.

Any of the above nucleic acid sequences encoding a peptide of the invention may consist of up to 30, 40, 50, 60, 70, 80, 90 or 100 nucleotides or more in length, depending on the length of the relevant peptide of the invention. Such nucleic acid sequences may include a nucleotide sequence having at least 70%, 75%, 80%, 85%, 90%, 95%, 99% identity, or 100% identity, to SEQ ID NOs: 10 to 13 or 16.

The nucleic acid construct of the second aspect may be part of an expression cassette. An expression cassette is a part of a vector. It comprises a promoter, an open reading frame and a 3′ untranslated region.

The nucleic acid construct of the second aspect of the invention may be in the form of a vector. A vector as used herein refers to a construct for introducing a nucleic acid sequence into a cell or a virus for expression or replication. It refers to a recombinant construct for example a plasmid, a virus or any other construct capable of expression or replication of the nucleic acid sequence upon introduction into a cell or virus.

Examples of vectors include, among others, chromosomal, episomal and virus-derived vectors. Generally, any vector suitable to maintain, propagate or express nucleic acid to express a polypeptide in a host, may be used for expression in this regard.

The nucleic acid constructs and vectors of the invention may be present within a cell. As used herein, a cell refers to a prokaryotic cell, such as a bacterial cell, or eukaryotic cell, such as an animal, plant or yeast cell.

### Diseases

The peptides, combination of peptides, nucleic acid constructs and/or the pharmaceutical compositions of the invention may be used in any method of therapy practised on the human or animal body.

The disease which can be treated/prevented by the peptides, compositions or methods of the present invention may in particular be any disease associated with abnormal or excessive angiogenesis. A wide range of such diseases is listed by Carmeliet (Nature Medicine 9, 653-660 (2003)). Examples include cancer, arthritis, psoriasis, asthma and atherosclerosis. Angiogenesis is also a feature of ocular disease and is a major cause of blindness. It is a significant contributing factor in diabetic retinopathy, exudative (wet) or nonexudative (dry) age related macular degeneration (AMD), corneal graft rejection, corneal neovascularization, retinopathy of prematurity (ROP), retinal artery or vein occlusion, neovascular glaucoma and sickle cell retinopathy. Accordingly, the disease which can be treated/prevented by the compositions or methods of the present invention can be any of these diseases.

### Pharmaceutical Compositions

In a third aspect the invention provides a pharmaceutical composition comprising a pharmaceutically acceptable carrier and a peptide, a fusion polypeptide, or a combination of peptides according to the first aspect or a nucleic acid according to the second aspect.

In another embodiment the pharmaceutical composition further comprises an anti-angiogenic compound. Examples of anti-angiogenic compounds are known in the art such as suramin, sorafenib and sunitinib.

The pharmaceutical compositions of the invention may be used in the treatment of diseases associated with excessive or abnormal angiogenesis for example cancer, arthritis, psoriasis, asthma, atherosclerosis and ocular diseases such as diabetic retinopathy, exudative (wet) or nonexudative (dry) macular degeneration (AMD), corneal graft rejection, corneal neovascularisation, neovascular glaucoma, retinopathy of prematurity (ROP), retinal artery or vein occlusion and sickle cell retinopathy. In an embodiment of the invention the pharmaceutical compositions of the invention are for use in the treatment of cancer.

A pharmaceutical composition according to the present invention may be presented in a form that is ready for immediate use. Alternatively, the composition may be presented in a form that requires some preparation prior to administration.

Pharmaceutical compositions of the invention may be adapted for administration by any appropriate route, for example by the oral (including buccal or sublingual), topical (including buccal, sublingual or transdermal), or parenteral (including subcutaneous, intramuscular, intravenous, intraperitoneal or intradermal) route.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents.

Excipients which may be used for injectable solutions include water, alcohols, polyols, glycerine and vegetable oils, for example. The compositions may be presented in unit-dose or multidose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the peptide or nucleic acid construct of the present invention.

When a combination of peptides described herein is administered in a method or medical use of the invention, the first and second peptide may be administered separately, sequentially or simultaneously.

### Treatment of Vascular Permeability

The invention also provides a peptide for use in treating diseases associated with vascular permeability.

The peptide may comprise an amino acid sequence having at least 70% identity to SEQ ID NO: 3 or SEQ ID NO: 4. The peptide may be as described in any of the embodiments set out for the first peptide in the combinations described in the “Peptide combinations” section above. The peptide may be administered as part of a combination described in the “Peptide combinations” section above.

The disease which can be treated/prevented by the peptide may be any disease associated with vascular permeability, i.e. comprising aberrant, increased, vascular permeability. Such diseases are well known in the art, as described for example in Park-Windhol & D'Amore (Annu Rev Pathol 2016 11:251-81), incorporated by reference herein, including any specific disorder of vascular permeability described therein. The disease may be associated with oedema and/or inflammation. A wide range of diseases are associated with vascular permeability, and examples include cancer, ocular diseases such as diabetic retinopathy and macular degeneration, such as early-stage AMD, rheumatoid arthritis, lymphoedema, asthma, ventilator induced-lung injury, acute lung injury, atherosclerosis, ischemic stroke, psoriasis, inflammatory bowel disorders, and myocardial infarction. The cancer may be a solid tumour. The cancer may be ovarian cancer or lung cancer, preferably epithelial ovarian cancer or nonsquamous non-small cell lung cancer. The cancer may comprise tumours that produce a large volume of fluid. The disease associated with vascular permeability may be refractory to treatment with an anti-VEGF antagonist.

In an embodiment, the peptide may be administered to a tumour having aberrant vascular permeability in combination with at least one chemotherapeutic agent. The aberrant vascular permeability of the tumour may result in surrounding fluid (oedema) reducing access of a chemotherapeutic agent to the tumour. The peptide may thus facilitate delivery of the chemotherapeutic agent to the tumour. The use of the peptide in combination with the at least one chemotherapeutic agent thus typically enhances the effect of the chemotherapeutic agent.

The peptide of the invention may be used in combination with any chemotherapeutic agent, such as any chemotherapeutic agent useful in treatment of a cancer of interest. The skilled person is able to select a chemotherapeutic agent suitable for treatment of a particular type of cancer.

Chemotherapeutic agents, such as anticancer agents, include: alkylating agents including including platinum coordination complexes such as cisplatin (cis-DDP) and carboplatin, nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including gemcitabine and folic acid analogues such as methotrexate (amethopterin), pemetrexed; pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

When a combination of peptides described herein is administered in a method or medical use of the invention where a chemotherapeutic agent is also administered, the first and second peptide may be administered separately, sequentially or simultaneously.

In an embodiment, the peptide may be administered to a tumour having aberrant vascular permeability in combination with irradiation treatment (radiotherapy). The irradiation treatment may comprise the use of a machine to direct radiation at a cancer or the use of a radiotherapy agent. The irradiation treatment may be thoracic irradiation treatment.

When a combination of peptides described herein is administered in a method or medical use of the invention where irradiation treatment, such as thoracic irradiation treatment, is also used, the first and second peptide may be administered separately, sequentially or simultaneously.

In an embodiment, the peptide may be administered to a tumour having aberrant vascular permeability in combination with a chemotherapeutic agent and radiotherapy. The radiotherapy may comprise the use of a machine to direct radiation at a cancer or the use of a radiotherapy agent.

When a combination of peptides described herein is administered in a method or medical use of the invention where a chemotherapeutic agent is also administered and radiotherapy is also used, the first and second peptide may be administered separately, sequentially or simultaneously. The peptides, compositions or methods of the present invention can be used to treat ocular diseases associated with vascular permeability that have not progressed to neovascularisation. The peptides, compositions or methods of the present invention can also prevent progression of ocular diseases associated with vascular permeability to an ocular disease comprising neovascularisation.

The peptides, compositions or methods of the present invention can thus be used to treat early-stage ocular diseases associated with vascular permeability. For example, peptides, compositions or methods of the present invention can treat early-stage AMD.

The first sign of AMD is a loss or slight impairment of central vision. Scanners can identify spots of drusen (fatty deposits) in the eye, a characteristic of the early stages of the disease. Thus, a patient comprising one or more of signs of early AMD can be treated with the peptides, compositions or methods of the present invention. The treatment may prevent progression to a later stage of AMD, such as a later stage comprising neovascularisation.

The peptides, compositions or methods of the present invention can also be used to treat diabetic retinopathy. Specifically, the present invention may be used to treat the disease at stages 1 (background retinopathy) or 2 (pre-proliferative retinopathy).

In stage 1 of diabetic retinopathy (background retinopathy), small bulges (microaneurysms) may appear in the blood vessels in the back of the eye (retina), which may leak small amounts of blood. This is common in patients with diabetes. At this stage, sight is not affected, although there is a higher risk of developing vision problems in the future. Treatment is not required, although care needs to be taken to prevent the problem getting worse. The chances of the disease progressing to the later Stages within 3 years are more than 25% if both eyes are affected.

In stage 2 (pre-proliferative retinopathy), more severe and widespread changes are seen in the retina, including bleeding into the retina. At this stage, there is a high risk that vision could eventually be affected. Patients are advised to have more frequent screening appointments (every 3 to 6 months) to monitor the eyes.

In stage 3 (proliferative retinopathy), new blood vessels and scar tissue will have formed on the retina, which can cause significant bleeding and lead to retinal detachment. At this stage, there is a very high risk of loss of vision. Treatment is offered to stabilise vision as much as possible, although it will not be possible to restore any vision that has already been lost.

Thus, a patient comprising one or more of signs of early diabetic retinopathy, such as a patient at stage 1 or 2 may be treated with the peptides, compositions or methods of the present invention. Such a treatment may prevent progression to a later stage of disease, such as a later stage comprising neovascularisation, for example stage 3.

### Methods of Treatment

In a fourth aspect the invention provides a method of treating a disease associated with (abnormal or excessive) angiogenesis or vascular permeability comprising administering to a subject in need thereof a therapeutically effective amount of a peptide or a combination of peptides according to the first aspect, a nucleic acid construct of the second aspect or a pharmaceutical composition of the third aspect.

A therapeutically effective amount may be a dose sufficient to reduce or inhibit angiogenesis. In an embodiment of the invention the method of treatment comprises administering a peptide, a combination of peptides, nucleic acid construct or pharmaceutical composition of the invention in combination with other anti-angiogenic therapy.

A therapeutically effective amount may be a dose sufficient to reduce or block vascular permeability. Vascular permeability is defined and can be evaluated according to the descriptions in the “Peptides” section above.

In an embodiment of the invention the method is for treating a disease such as cancer, arthritis, psoriasis, asthma, atherosclerosis and ocular diseases such as diabetic retinopathy, exudative (wet) or nonexudative (dry) macular degeneration (AMD), corneal graft rejection, corneal neovascularisation, retinopathy of prematurity (ROP), neovascular glaucoma, retinal artery or vein occlusion and sickle cell retinopathy. In a further embodiment the method is for treating cancer. As used herein, a subject refers to an animal, including a human being. An animal can include mice, rats, fowls such as chicken, ruminants such as cows, goat, deer, sheep and other animals such as pigs, cats, dogs and primates such as humans, chimpanzees, gorillas and monkeys. Preferably the subject is human.

This aspect of the invention also extends to:
A peptide, a fusion polypeptide, or a combination of peptides according to the first aspect, a nucleic acid construct of the second aspect or a pharmaceutical composition of the third aspect for use in a method of therapy practised in the human or animal body.
A peptide, a fusion polypeptide, or a combination of peptides according to the first aspect, a nucleic acid construct of the second aspect or a pharmaceutical composition of the third aspect for use in the treatment of a disease associated with angiogenesis.
Use of a peptide, a fusion polypeptide, or a combination of peptides according to the first aspect for the manufacture of a medicament for treatment of a disease associated with angiogenesis.
A peptide for use in a method of treatment of a disease associated with vascular permeability.
Use of a peptide for the manufacture of a medicament for treatment of a disease associated with vascular permeability.

As used herein, “treatment” is also intended to cover preventative treatment, i.e. prophylaxis.

### Dosages

A therapeutically effective amount is the dose sufficient to reduce or inhibit angiogenesis and/or vascular permeability.

Doses for delivery and administration can be based upon current existing protocols, empirically determined, using animal disease models or optionally in human clinical trials. Initial study doses can be based upon animal studies set forth herein, for a mouse, for example.

Doses can vary and depend upon whether the treatment is prophylactic or therapeutic, the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled person will appreciate the factors that may influence the dosage and timing required to provide an amount sufficient for providing a therapeutic or prophylactic benefit.

### Kits of the Invention

In a fifth aspect the invention provides a kit of parts comprising peptides, nucleic acid constructs and/or pharmaceutical compositions of the invention.

In an embodiment of the invention the kit is for use in the treatment of diseases associated with angiogenesis. In a preferred embodiment the kit is for use in the treatment of cancer or ocular diseases.

The kit may include a sealed container containing the peptide of the invention as a lyophilized powder and a second container containing a solvent. The peptide may be freeze dried. Further components may be included with the solid or liquid part. Thus the kit may comprise a first container containing the peptide and a second containing isotonic saline, or a first container containing the peptide and mannitol and a second container containing sterile water. Prior to administration the solvent is added to the container containing solid component in order to give the solution for injection.

### EXAMPLES

### Materials and Methods

### Syndecan-3 Ectodomain GST Fusion Proteins

The full length syndecan-3 cDNA was obtained from Source BioScience. The entire length of the mature syndecan-3 ectodomain (A45-L380) was amplified by PCR using the primers S3forEcoRI (ttaattgaattcgctcaacgctggcgcaatg) and S3revHindIII (ttaattaagcttctacagtatgctcttctgaggga) (Integrated DNA Technologies) and the resultant product was digested with EcoRI and HIndIII and ligated into the equivalent sites of pET41 (Novagen) according to Manufacturer's instructions. Truncated versions of the murine syndecan-3 ectodomian sequence were generated such that anti-angiogenic region could be mapped. Plasmids were generated as above to incorporate A45-A184, P195-L380, L91-V310, E151-V310, E151-V320, and P195-A221 in pET41 and thus generate truncated GST fusion proteins for further analysis. Plasmids were verified by sequencing and transformed into the BL21 strain of Escherichia coli (Novagen). S3ED proteins were purified from bacterial cultures which had reached an OD600 of 0.4 prior to the addition of 0.1 M Isopropyl β-D-1-thiogalactopyranoside (IPTG) and subsequent outgrowth for 4 hours. Affinity purification of both GST and S3ED was performed using glutathione-sepharose 4B (GE Healthcare) as described by the manufacturers.

### Peptides

Peptides were synthesized by Cambridge Peptides and reconstituted in PBS at a concentration of 100 μM.

### Scratch Wound Cell Migration Assay

Confluent monolayers of HUVECs were scratched with a pipette tip, cells were then washed twice with PBS prior to the addition of growth medium supplemented with either GST fusion proteins or peptide treatments. Wounds were monitored by time lapse microscopy using an Olympus IX81 Microscope Hamamatsu Orca ER digital camera. Regions of interests in the scratch area were recorded using Cell\hat{ }M software (Olympus) and micrographs of ROIs taken every 30 min for 16 hours with an Olympus IX81 inverted microscope. The initial and final gap was measured using ImageJ software (NIH).

### Ex-Vivo Angiogenic Sprouting Assays

Thoracic aortas were dissected from either male wistar rats (˜200 g) or 4-5 week old male C57BL6J mice (both obtained from Charles River, UK). The fat surrounding the aortas was removed as were any branches and the tissue sliced into <1 mm rings. Choroid membranes explants were isolated from 21 to 28 day old male C57BL6J mice. Eyes were punctured with scissors and the iris/cornea/lens and retina removed. The resultant Choroid was then cut into 1 mm3 chunks. After dissection both aortic rings and choroid explants were incubated overnight at 37° C. in Opti-MEM (Thermo Fisher Scientific) prior to embedding on ice in 0.15 ml of 1 mg/ml Collagen I (Millipore) in E4 medium (Thermo Fisher Scientific) in 48 well plates (Corning). After 30 mins incubation at 37° C., to set the Collagen, wells were supplemented with 200 ul Opti-MEM with 1% FBS. For rat aortic rings 10 ng/ml of VEGA (R and D Systems) was added, for mouse aortic and choroid explants 30 ng/ml was used. Treatments at the concentrations indicated were also added to this and media replenished every 3 days. Angiogenic sprouts from explants were counted after 7 days and expressed as sprouts/ring or explant.

### Oxygen-Induced Retinopathy (OIR) Mouse Model

Neonatal mice (both male and female) at P7 were exposed to 75% oxygen for 5 days with their nursing mothers. At P12, they were returned to normoxia. Animals were euthanized at P12 to determine the area of vaso-obliteration or at P17 to determine the rate of retinal revascularization and pre-retinal neovascularization. As postnatal weight gain has been shown to affect outcome in the OIR model, only weight matched (±1 g) pups were used in each experiment. Sub retinal injections of the treatments described and controls was administered at day P12 upon return to normoxic conditions. Analysis of retinal vasculature was done as previously described. Briefly, eyes were enucleated, fixed with 4% PFA for one hour and retinas dissected. Flat mount retinas were blocked in 10% normal goat serum and 10% fetal bovine serum for 2 hours, incubated overnight with isolectin GS-IB4 (1:100). Briefly, retinas were imaged using confocal microscopy (Carl Zeiss LSM 700) with 10× objective. Pre-retinal neovascular tufts were readily distinguished from the superficial vascular plexus by focusing just above the inner limiting membrane. Areas of vascular obliteration and pathological neovascularization (neovascular tufts) were quantified using Adobe Photoshop CS3.

### Evans Blue Assay for Dermal Vascular Permeability

Male C57BL6J mice (6-8 weeks old) were anesthetized by i.m. injection of 1 ml/kg ketamine (40 mg) and xylazine (2 mg) in saline solution. The fur on the animals back was shaved with an electric razor prior to intravenous administration of Evans Blue dye (0.5% in PBS, 5 μl per g bodyweight) via the tail vein. Sub-cutaneous injections consisting of 50 μl of PBS with either 100 ng of VEGFA (R and D Systems) or 100 μg of Bradykinin or PBS alone with or without QM107 dose were administered to the mouse dorsal skin. After 90 min animals were sacrificed by cervical dislocation. Dorsal skin was removed and injected sites were excised as circular patches using a metal punch. (˜8 mm in diameter). Samples were then incubated in 250 μl of formamide at 56° C. for 24 h to extract Evans Blue dye from the tissues. The amount of accumulated Evans Blue dye was quantified by spectroscopy at 620 nm using a Spectra MR spectrometer (Dynex technologies Ltd., West Sussex, UK). Results are presented as the optical density at 620 nm (OD620) per mg tissue and per mouse.

### Example 1: The Syndecan-3 Extracellular Core Protein Inhibits Angiogenic Sprout Formation from Rat Aortic Rings

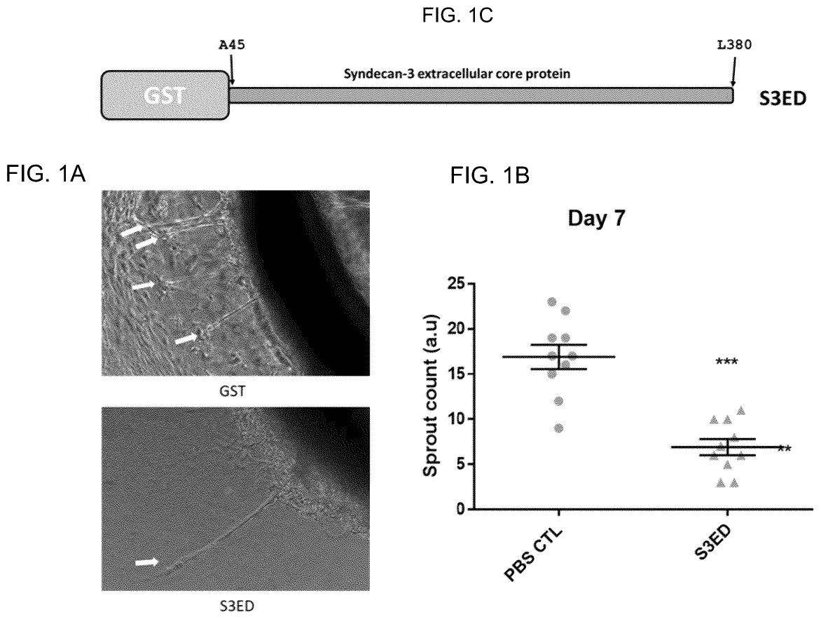

The full length of murine SDC3 (A45-L380) fused at the N-terminus to Glutathione-S-transferase (GST) in bacteria (S3ED, FIG. 1) was expressed. The GST fusion enabled the protein to remain soluble and allow ease of purification. Bacteria do not possess the necessary cellular machinery to generate GAG chains, and also the O-linked sugars which are predicated to occur on the full length molecule expressed in mammalian cells. In a first set of experiments (FIGS. 1A and B) we showed that GST-S3ED inhibits angiogenic spout formation from rat aortic explants as compared to the GST control where angiogenesis is not affected. This data was surprising since SDC3 has long been associated exclusively with cells of a neuronal lineage and roles in angiogenesis and endothelial cell biology had not been identified.

### Example 2: Miniaturization Strategy to Identify Minimum Peptide Sequence for Inhibition of Angiogenesis

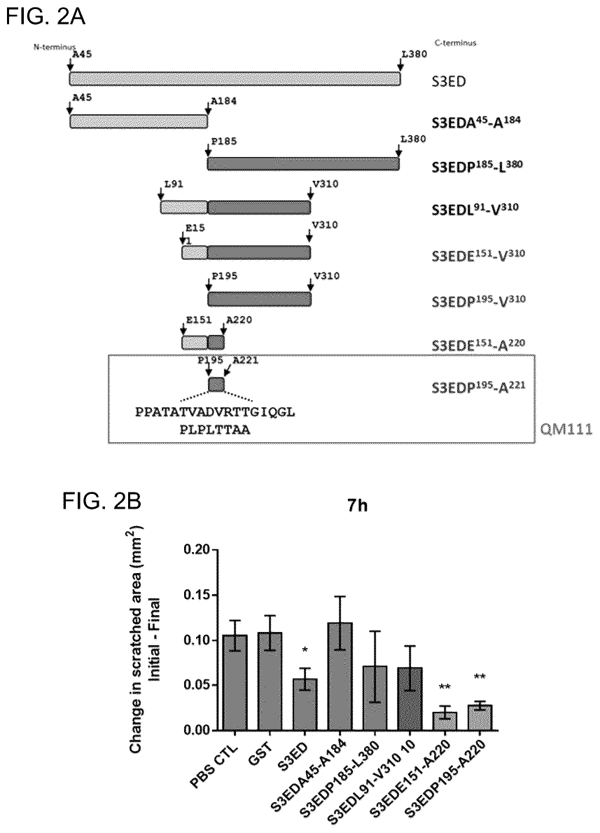

There is little or no sequence conservation between the extracellular core proteins of the 4 syndecan family members, and no way of predicting which region of murine SDC3 contained its antiangiogenic properties. An iterative approach was therefore adopted in which mutagenesis was performed on S3ED such that it was truncated at different points along its length (FIG. 2A). These truncated forms of S3ED were tested in scratch wound endothelial cell migration assays as a readout of anti-angiogenic activity. This revealed that the anti-angiogenic activity of S3ED resides between P195 and A220 of the murine sequence (FIG. 2B).

### Example 3: Peptides Corresponding to the Anti-Angiogenic Regions of Human and Murine Syndecan-3 Inhibit HUVEC Cell Migration and have Comparable Efficacy to QM107

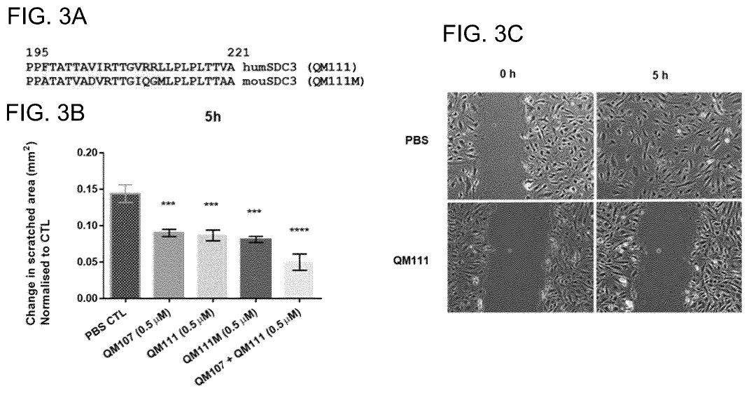

Experiments were conducted to test whether peptides corresponding to these 27 amino acids from the murine sequence retained the activity of full length S3ED and whether the corresponding region of the human sequence also retained these properties (FIG. 3A). The use of the human sequence may be advantageous in that it is a naturally occurring molecule within humans and the likelihood of adverse immune responses reduced. Both human (Developmental name QM111) and the mouse peptide (QM111M) inhibited endothelial cell migration to the same degree (FIGS. 3A and B). Of note both SDC3 derived peptides performed as well as QM107 and when QM111 and QM107 were used in conjunction endothelial cell migration was inhibited to a greater degree.

### Example 4: Peptides Corresponding to the Anti-Angiogenic Regions of Human and Murine Syndecan-3 Inhibit Angiogenic Sprout Formation and have Comparable Efficacy to QM107

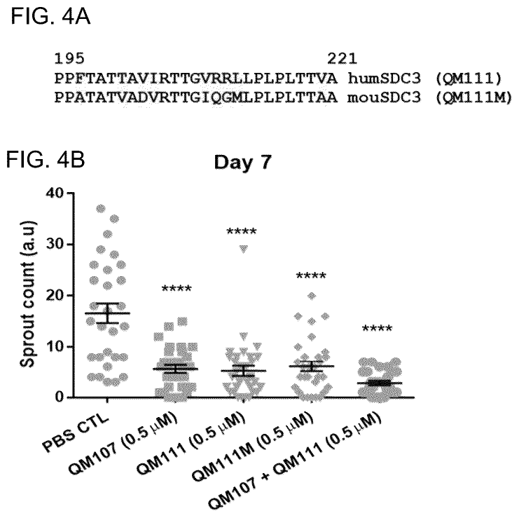

In further experiments the peptides corresponding to the 27 amino acids from the murine sequence were tested in a more physiologically relevant model looking at angiogenic sprout formation from ex vivo aortic explants. Both human and mouse sequences inhibited angiogenic sprout formation to the same degree as QM107 (FIGS. 4A and B), and as previously a combination of QM111 and QM107 gave a more robust response.

### Example 5: QM111 Blocks Pathological Neovascularization in a Model of Diabetic Retinopathy

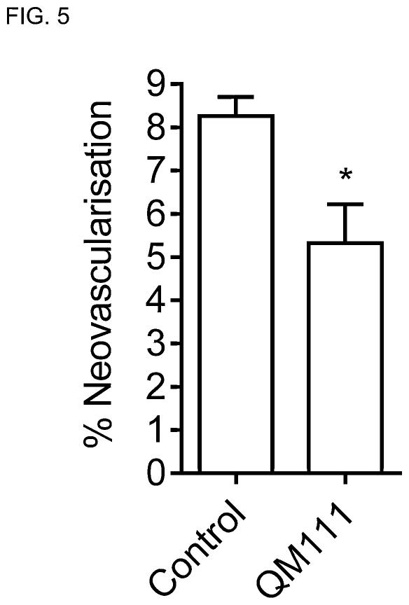

Oxygen induced retinopathy recapitulates many features of diabetic retinopathy. Neonatal pups and mothers were exposed to a hyperoxic environment (80% Oxygen) at day p7 for 5 days prior to a return to normoxia. This obliterates the developing retinal vasculature and initiates a hypoxic response causing an aberrant pathological angiogenesis, as evidenced by the formation of neovascular tufts. To obtain proof of concept that QM111 could block neovascularisation in vivo 0.5 μM of QM111 was administered to pups by intra retinal injection upon their return to normoxic conditions. Analysis of the retinal vasculature at this point revealed that there was reduced pathological angiogenesis in animals treated with QM111 (FIG. 5).

### Example 6: Miniaturization Strategy Based on Comparison between Mouse and Human Sequences

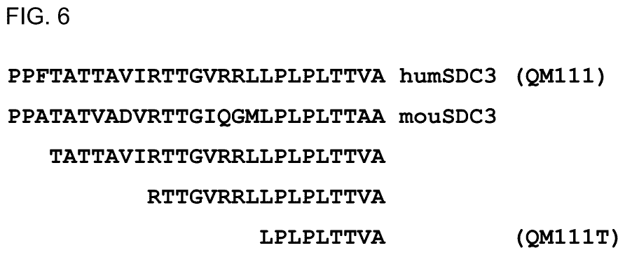

The sequence of QM111 is 28aa. Experiments seeking to miniaturise QM111 were performed. Comparison of mouse and human peptide sequences revealed that the C-terminal 9 amino acids were conserved with the exception of 1 amino acid (FIG. 6). Therefore this smaller version of QM111 (Developmental name QM111T) was synthesised.

### Example 7: QM111T Inhibits Choroidal Neovascularization

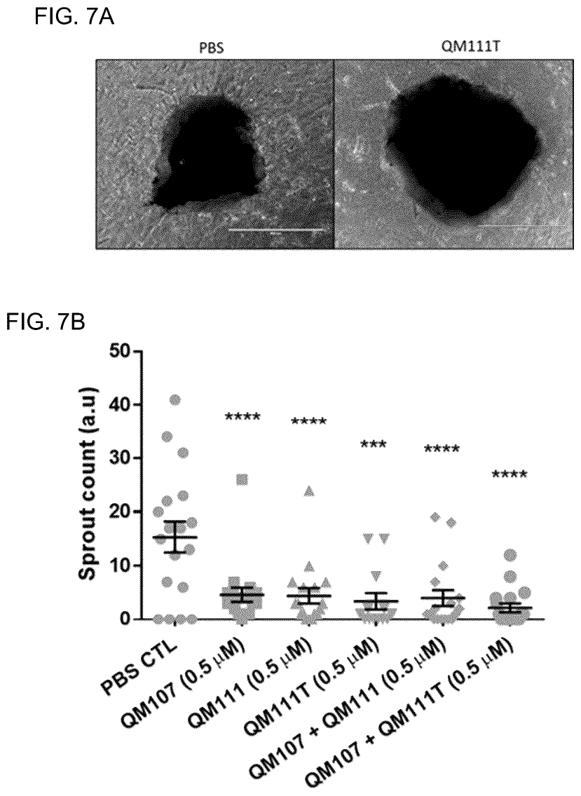

QM111T was then tested to see whether it retained the anti-angiogenic properties of QM111 in an ex vivo assay in which angiogenic sprouts are measured from fragments from the choroid membranes of murine eyes. This model has relevance to the pathology of Wet Age Related Macular Degeneration. QM111T inhibited angiogenesis to the same degree as QM111 and QM107 and as previously observed gave a more robust response when used in combination with QM107 (FIG. 7).

### Example 8: Combination Therapy using QM107 and QM111 Results in Less Variability in Angiogenesis Assays

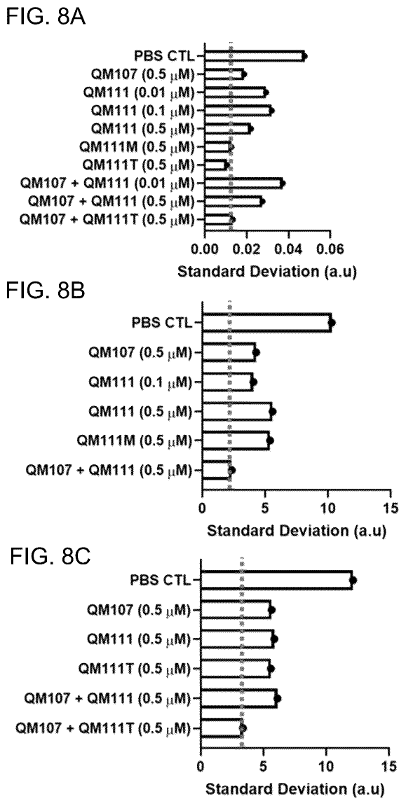

In experiments in which QM107 and QM111 or QM111T have been tested, a reduction in the variability of the inhibitory response has been observed, suggesting that a combination therapy may offer a more effective therapeutic outcome in patients. In order to try to visualise this, the standard deviation from experimental replicates from FIGS. 3, 4 and 7 (Examples 3, 4 and 7) were calculated. In all 3 instances (EC migration, FIG. 8A; Aortic rings, FIG. 8B and Choroid explant, FIG. 8C), the standard deviation was greatly reduced when QM107 and QM111 were used in combination (see red lines on graphs). This data suggests that using both peptides together may offer a more effective treatment strategy than using each one alone.

### Example 9: QM107 Blocks Vascular Permeability Responses to VEGFA and Bradykinin

Blood vessels are comprised of a single layer of endothelial cells which are bound tightly together by a complex assemblage of molecules collectively termed endothelial junctions. These junctions are essential for vascular integrity and the main function of blood vessels which is to carry blood around the body. For new blood vessels to form a critical early stage is the disassembly of endothelial cell junctions such that the cells can migrate, proliferate and form a new vessel. Endothelial cell junctions can also become disrupted in response to inflammatory mediators and can result in vascular oedema, which manifests as swelling.

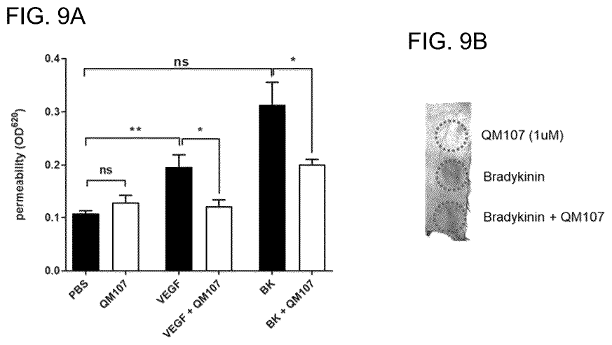

Experiments were performed to determine whether QM107 has any effect on vascular permeability. To this end a model of dermal hyperpermeability was used in which the leakage of Evan's Blue dye from murine dorsal skin microvasculature in response to known stimulators of this process, bradykinin and VEGFA, was measured. Both VEGFA and Bradykinin stimulated a strong vascular permeability response (FIG. 9). Strikingly, however, in both cases the response is ablated when these treatments are co injected with QM107.

Sequences of the invention

SEQ ID NO: 1-minimal active amino acid sequence of human
Syndecan-3 (SDC3)
LPLPLTTVA
SEQ ID NO: 2-longer active amino acid sequence of human
SDC3
PPFTATT AVIRTTGVRR LLPLPLTTVA
SEQ ID NO: 3-amino acid sequence of residues 123-140 of
human Syndecan-2 (SDC2)
PAEEDTNV YTEKHSDSLF
SEQ ID NO: 4-amino acid sequence of residues 124-141 of
mouse SDC2
PAIKSTD VYTEKHSDNL F
SEQ ID NO: 5-amino acid sequence of human SDC2
MRRAWILLTL GLVACVSAES RAELTSDKDM YLDNSSIEEA SGVYPIDDDD
YASASGSGAD EDVESPELTT SRPLPKILLT SAAPKVETTT LNIQNKIPAQ
TKSPEETDKE KVHLSDSERK MDPAEEDTNV YTEKHSDSLF KRTEVLAAVI
AGGVIGFLFA IFLILLLVYR MRKKDEGSYD LGERKPSSAA YQKAPTKEFY
A
SEQ ID NO: 6-amino acid sequence of mouse SDC2
MQRAWILLTL GLMACVSAET RTELTSDKDM YLDNSSIEEA SGVYPIDDDD
YSSASGSGAD EDIESPVLTT SQLIPRIPLT SAASPKVETM TLKTQSITPA
QTESPEETDK EEVDISEAEE KLGPAIKSTD VYTEKHSDNL FKRTEVLAAV
IAGGVIGFLF AIFLILLLVY RMRKKDEGSY DLGERKPSSA AYQKAPTKEF
YA
SEQ ID NO: 7-active amino acid sequence fragment of mouse
SDC3
LPLPLTTA A
SEQ ID NO: 8-amino acid sequence of human SDC3
MKPGPPHRAG AAHGAGAGAG AAAGPGARGL LLPPLLLLLL AGRAAGAQRW
RSENFERPVD LEGSGDDDSF PDDELDDLYS GSGSGYFEQE SGIETAMRFS
PDVALAVSTT PAVLPTTNIQ PVGTPFEELP SERPTLEPAT SPLVVTEVPE
EPSQRATTVS TTMATTAATS TGDPTVATVP ATVATATPST PAAPPFTATT
AVIRTTGVRR LLPLPLTTVA TARATTPEAP SPPTTAAVLD TEAPTPRLVS
TATSRPRALP RPATTQEPDI PERSTLPLGT TAPGPTEVAQ TPTPETFLTT
IRNEPEVPVS GGPSGDFELP EEETTQPDTA NEVVAVGGAA AKASSPPGTL
PKGARPGPGL LDNAIDSGSS AAQLPQKSIL ERKEVLVAVI VGGVVGALFA
AFLVTLLIYR MKKKDEGSYT LEEPKQASVT YQKPDKQEEF YA
SEQ ID NO: 9-amino acid sequence of mouse SDC3
MKPGPPRRGT AQGQRVDTAT HAPGARGLLL PPLLLLLLAG RAAGAQRWRN
ENFERPVDLE GSGDDDSFPD DELDDLYSGS GSGYFEQESG LETAMRFIPD
MALAAPTAPA MLPTTVIQPV DTPFEELLSE HPRPEPVTSP PLVTEVKEVV
EESSQKATTI STTTSTTAAT TTGAPTMATA PATAATTAPS TPEAPPATAT
VADVRTTGIQ GMLPLPLTTA ATAKITTPAA PSPPTTVATL DTEAPTPRLV
NTATSRPQSL PRPITTQEPE VAERSTLPLG TTAPGPTEVA QTPTPESLLT
TTQDEPEVPV SGGPSGDFEL QEETTQPDTA NEVVAVEGAA AKPSPPLGTL
PKGARPGLGL HDNAIDSGSS AAQLLQKSIL ERKEVLVAVI VGGVVGALFA
AFLVTLLIYR MKKKDEGSYT LEEPKQASVT YQKPDKQEEF YA
SEQ ID NO: 10-nucleotide sequence encoding for active amino
acid sequence fragment of human SDC3
CTGCCTCTCC CACTGACCAC AGTGGC
SEQ ID NO: 11-nucleotide sequence of the longer active
amino acid sequence of human SDC3
CCTGCAGCAC CCCCTTTTAC GGCCACCACT GCTGTTATAA GGACCACTGG
CGTACGGAGG CTTCTGCCTC TCCCACTGAC CACAGTGGCT
SEQ ID NO: 12-nucleotide sequence for residues 123-140 of
human SDC2
CCAG CCGAAGAGGA TACAAATGTG TATACTGAGA AACACTCAGA CAGTCTGTTT
SEQ ID NO: 13-nucleotide sequence for residues 124-141 of
mouse SDC2
C CTGCTATAAA AAGCACAGAT GTGTACACGG AGAAACATTC AGACAATCTG TTT
SEQ ID NO: 14-nucleotide sequence of human SDC2
ATGCGGCGCG CGTGGATCCT GCTCACCTTG GGCTTGGTGG CCTGCGTGTC
GGCGGAGTCG AGAGCAGAGC TGACATCTGA TAAAGACATG TACCTTGACA
ACAGCTCCAT TGAAGAAGCT TCAGGAGTGT ATCCTATTGA TGACGATGAC
TACGCTTCTG CGTCTGGCTC GGGAGCTGAT GAGGATGTAG AGAGTCCAGA
GCTGACAACA TCTCGACCAC TTCCAAAGAT ACTGTTGACT AGTGCTGCTC
CAAAAGTGGA AACCACGACG CTGAATATAC AGAACAAGAT ACCTGCTCAG
ACAAAGTCAC CTGAAGAAAC TGATAAAGAG AAAGTTCACC TCTCTGACTC
AGAAAGGAAA ATGGACCCAG CCGAAGAGGA TACAAATGTG TATACTGAGA
AACACTCAGA CAGTCTGTTT AAACGGACAG AAGTCCTAGC AGCTGTCATT
GCTGGTGGAG TTATTGGCTT TCTCTTTGCA ATTTTTCTTA TCCTGCTGTT
GGTGTATCGC ATGAGAAAGA AGGATGAAGG AAGCTATGAC CTTGGAGAAC
GCAAACCATC CAGTGCTGCT TATCAGAAGG CACCTACTAA GGAGTTTTAT
GCGTAA
SEQ ID NO: 15-nucleotide sequence of mouse SDC2
ATGCAGCGCG CGTGGATCCT GCTCACCTTG GGCTTGATGG CCTGTGTGTC
CGCAGAGACG AGAACAGAGC TGACATCCGA TAAGGATATG TACCTTGACA
ATAGCTCCAT TGAGGAAGCT TCAGGAGTAT ATCCTATTGA TGATGATGAC
TATTCTTCTG CCTCAGGCTC AGGAGCTGAT GAAGACATAG AGAGTCCAGT
TCTGACAACA TCCCAACTGA TTCCAAGAAT CCCACTCACT AGTGCTGCTT
CCCCCAAAGT GGAAACCATG ACGTTGAAGA CACAAAGCAT TACACCTGCT
CAGACTGAGT CACCTGAAGA AACTGACAAG GAGGAAGTTG ACATTTCTGA
GGCAGAAGAG AAGCTGGGCC CTGCTATAAA AAGCACAGAT GTGTACACGG
AGAAACATTC AGACAATCTG TTTAAACGGA CAGAAGTTCT AGCAGCCGTC
ATTGCTGGTG GTGTGATCGG CTTTCTCTTT GCCATTTTCC TCATCCTGCT
ATTGGTGTAC CGCATGCGGA AGAAAGATGA AGGAAGCTAC GACCTTGGAG
AACGCAAACC ATCCAGCGCA GCTTACCAGA AGGCACCCAC TAAGGAGTTT
TATGCATAA
SEQ ID NO: 16-nucleotide sequence encoding for active amino
acid sequence fragment of mouse SDC3
CGCCCCCTGC CACGGCTACC GTGGCTGACG TAAGGACCAC CGGCATACAG
GGGATGCTGC CTCTTCCCCT GACCACAGCT GCC
SEQ ID NO: 17-nucleotide sequence of human SDC3
ATGAAGCCGG GGCCGCCGCA CCGTGCCGGG GCCGCCCACG GGGCCGGCGC
CGGGGCCGGG GCCGCGGCCG GGCCCGGGGC CCGCGGGCTG CTCCTGCCAC
CGCTGCTGCT GCTGCTGCTG GCGGGGCGCG CCGCGGGGGC CCAGCGCTGG
CGCAGTGAGA ACTTCGAGAG ACCCGTGGAC CTGGAGGGCT CTGGGGATGA
TGACTCCTTT CCCGATGATG AACTGGATGA CCTCTACTCG GGGTCGGGCT
CGGGCTACTT CGAGCAGGAG TCGGGCATTG AGACAGCCAT GCGCTTCAGC
CCAGATGTAG CCCTGGCGGT GTCCACCACA CCTGCGGTGC TGCCCACCAC
GAACATCCAG CCTGTGGGCA CACCATTTGA AGAGCTCCCC TCTGAGCGCC
CCACCCTGGA GCCAGCCACC AGCCCCCTGG TGGTGACAGA AGTCCCGGAA
GAGCCCAGCC AGAGAGCCAC CACCGTCTCC ACTACCATGG CTACCACTGC
TGCCACAAGC ACAGGGGACC CGACTGTGGC CACAGTGCCT GCCACAGTGG
CCACCGCCAC CCCCAGCACC CCTGCAGCAC CCCCTTTTAC GGCCACCACT
GCTGTTATAA GGACCACTGG CGTACGGAGG CTTCTGCCTC TCCCACTGAC
CACAGTGGCT ACGGCACGGG CCACTACCCC CGAGGCGCCC TCCCCGCCCA
CCACGGCGGC TGTCTTGGAC ACCGAGGCCC CAACACCCAG GCTGGTCAGC
ACAGCTACCT CCCGGCCAAG AGCCCTTCCC AGGCCGGCCA CCACCCAGGA
GCCTGACATC CCTGAGAGGA GCACCCTGCC CCTGGGGACC ACTGCCCCTG
GACCCACAGA GGTGGCTCAG ACCCCAACTC CAGAGACCTT CCTGACCACA
ATCCGGGATG AGCCAGAGGT TCCGGTGAGT GGGGGGCCCA GTGGAGACTT
CGAGCTGCCA GAAGAAGAGA CCACACAACC AGACACAGCC AATGAGGTGG
TAGCTGTGGG AGGGGCTGCG GCCAAGGCAT CATCTCCACC TGGGACACTG
CCCAAGGGTG CCCGCCCGGG CCCTGGCCTC CTGGACAATG CCATCGACTC
GGGCAGCTCA GCTGCTCAGC TGCCTCAGAA GAGTATCCTG GAGCGGAAGG
AGGTGCTCGT AGCTGTGATT GTGGGGGGGG TGGTGGGCGC CCTCTTTGCT
GCCTTCTTGG TCACACTGCT CATCTATCGT ATGAAGAAAA AGGATGAGGG
CAGCTACACG CTGGAGGAAC CCAAGCAGGC GAGCGTCACA TACCAGAAGC
CTGACAAGCA GGAGGAGTTC TATGCCTAG
SEQ ID NO: 18-nucleotide sequence of mouse SDC3
ATGAAGCCCG GGCCGCCGCG CCGCGGGACC GCACAGGGGC AGCGCGTGGA
CACCGCCACC CATGCGCCCG GGGCCCGCGG GCTGTTGCTG CCACCGCTGC
TGCTGCTGCT GCTGGCCGGC CGCGCCGCGG GGGCTCAACG CTGGCGCAAT
GAGAACTTCG AGAGGCCGGT GGATCTTGAG GGCTCAGGGG ATGACGACTC
GTTTCCTGAT GATGAACTAG ACGACCTCTA CTCGGGGTCA GGCTCTGGCT
ACTTCGAGCA GGAGTCCGGC CTTGAGACGG CCATGCGGTT CATCCCTGAT
ATGGCCCTGG CTGCGCCCAC TGCACCTGCC ATGCTACCCA CAACCGTTAT
CCAGCCCGTG GACACCCCAT TTGAGGAACT CCTTTCTGAG CACCCCAGCC
CTGAACCAGT CACCAGTCCC CCGCTGGTGA CAGAGGTGAC AGAGGTCGTA
GAAGAGTCCA GCCAGAAAGC TACCACCATC TCTACCACCA CATCTACCAC
CGCGGCCACC ACCACAGGGG CCCCAACTAT GGCCACAGCA CCTGCCACAG
CAGCCACCAC TGCCCCTAGC ACTCCCGAGG CGCCCCCTGC CACGGCTACC
GTGGCTGACG TAAGGACCAC CGGCATACAG GGGATGCTGC CTCTTCCCCT
GACCACAGCT GCCACAGCCA AGATCACTAC CCCAGCAGCA CCCTCACCAC
CCACTACTGT GGCTACCTTG GACACAGAGG CCCCGACACC TAGGCTGGTC
AACACAGCTA CCTCGAGGCC ACGAGCCCTT CCTCGGCCAG TCACCACCCA
GGAGCCTGAT GTTGCTGAGA GGAGTACCCT GCCGTTGGGG ACCACGGCTC
CTGGACCCAC GGAGATGGCT CAGACCCCAA CTCCAGAGTC CCTTCTGACC
ACCATCCAGG ATGAGCCAGA GGTGCCAGTA AGTGGGGGGC CCAGCGGGGA
CTTTGAGCTT CAAGAAGAGA CCACGCAGCC GGACACGGCC AATGAGGTGG
TGGCTGTGGA AGGAGCCGCG GCCAAGCCGT CACCTCCACT GGGGACACTG
CCCAAGGGTG CCCGCCCAGG CCCTGGCCTC CACGACAATG CCATCGATTC
GGGCAGCTCG GCCGCCCAGC TCCCTCAGAA GAGCATACTG GAGCGGAAGG
AGGTGCTCGT AGCCGTGATC GTGGGTGGGG TGGTGGGCGC CCTCTTCGCT
GCCTTCCTGG TCACGCTGCT CATCTACCGC ATGAAGAAGA AGGACGAAGG
CAGCTACACC TTGGAAGAAC CCAAGCAGGC AAGCGTCACG TACCAGAAAC
CTGACAAGCA GGAGGAGTTC TACGCTTAG
SEQ ID NO: 19-Fusion of minimal active amino acid sequence
of human Syndecan-3 (SDC3) and amino acid sequence of
residues 123-140 of human Syndecan-2 (SDC2)-residues 123-
140 of human Syndecan-2 (SDC2) bound to C-terminus of minimal
active amino acid sequence of human Syndecan-3 (SDC3)
LPLPLTTVAPAEEDTNVYTEKHSDSLF
SEQ ID NO: 20-Fusion of minimal active amino acid sequence
of human Syndecan-3 (SDC3) and amino acid sequence of
residues 123-140 of human Syndecan-2 (SDC2)-residues 123-
140 of human Syndecan-2 (SDC2) bound to N-terminus of minimal
active amino acid sequence of human Syndecan-3 (SDC3)
PAEEDTNVYTEKHSDSLFLPLPLTTVA
SEQ ID NO: 21-Fusion of minimal active amino acid sequence
of human Syndecan-3 (SDC3) and amino acid sequence of
residues 123-140 of human Syndecan-2 (SDC2)-residues 123-
140 of human Syndecan-2 (SDC2) bound to C-terminus of minimal
active amino acid sequence of human Syndecan-3 (SDC3) via
linker
LPLPLTTVAGGSPAEEDTNVYTEKHSDSLF
SEQ ID NO: 22-Fusion of minimal active amino acid sequence
of human Syndecan-3 (SDC3) and amino acid sequence of
residues 123-140 of human Syndecan-2 (SDC2)-residues 123-
140 of human Syndecan-2 (SDC2) bound to N-terminus of minimal
active amino acid sequence of human Syndecan-3 (SDC3) via
linker
PAEEDTNVYTEKHSDSLFGGSLPLPLTTVA

1. A peptide comprising an amino acid sequence having at least 70% identity to SEQ ID NO: 1.

2. The peptide according to claim 1, wherein the peptide comprises the amino acid sequence of SEQ ID NO: 2 or 7.

3. The peptide according to claim 1, which is of up to 50 amino acids in length.

4. A fusion polypeptide comprising the peptide according to any one of claims 1 to 3 fused to a heterologous peptide

5. The fusion peptide of claim 4, wherein the heterologous peptide comprises an amino acid sequence having at least 70% identity to SEQ ID NO: 3 or SEQ ID NO: 4, optionally wherein the fusion peptide comprises the amino acid sequence of SEQ ID NO: 19 or SEQ ID NO: 20.

6. The fusion peptide of claim 4, wherein the peptide according to any one of claims 1 to 3 is fused to the heterologous peptide using a linker, optionally wherein the linker is a peptide linker.

7. The fusion peptide of claim 4 or 6, wherein the heterologous peptide comprises an amino acid sequence having at least 70% identity to SEQ ID NO: 3 or SEQ ID NO: 4, optionally wherein the fusion peptide comprises the amino acid sequence of SEQ ID NO: 21 or SEQ ID NO: 22.

8. A combination of two peptides, the first peptide comprising an amino acid sequence having at least 70% identity to SEQ ID NO: 3 or SEQ ID NO: 4, the second peptide comprising an amino acid sequence having at least 70% identity to SEQ ID NO: 1.

9. The combination according to claim 8, wherein the first peptide consists of up to 25 amino acids and includes an amino acid sequence having at least 70% identity to SEQ ID NO: 3 or SEQ ID NO: 4, optionally wherein the first peptide comprises or consists of an amino acid sequence having at least 70% identity to up to 25 consecutive amino acid residues selected from: amino acid residues 120-144 of SEQ ID NO: 5 or amino acid residues 121-145 of SEQ ID NO: 6.

10. The combination according to claim 9, wherein the first peptide consists of an amino acid sequence having at least 70% identity to SEQ ID NO: 3 or SEQ ID NO: 4.

11. A nucleic acid construct encoding a peptide according to any one of claims 1 to 3, a fusion polypeptide according to claims 4 to 7, or a combination of peptides according to any one of claims 8 to 10.

12. A vector comprising a nucleic acid construct according to claim 11.

13. A cell comprising a nucleic acid construct according to claim 11 or a vector according to claim 12.

14. A pharmaceutical composition comprising a pharmaceutically acceptable carrier and a peptide according to any one of claims 1 to 3, a fusion polypeptide according to claims 4 to 7, or a combination of peptides according to any one of claims 8 to 10, or a nucleic acid construct according to claim 11.

15. A peptide according to any one of claims 1 to 3, a fusion polypeptide according to claims 4 to 7, a combination of peptides according to any one of claims 8 to 10, a nucleic acid construct according to claim 11 or a pharmaceutical composition according to claim 14, for use in a method of therapy practised on the human or animal body.

16. A peptide according to any one of claims 1 to 3, a fusion polypeptide according to claims 4 to 7, a combination of peptides according to any one of claims 8 to 10, a nucleic acid construct according to claim 11 or a pharmaceutical composition according to claim 14, for use in a method of treatment of a disease associated with angiogenesis.

17. A peptide, combination, nucleic acid construct or pharmaceutical composition for use according to claim 16, wherein the disease is cancer, arthritis, psoriasis, asthma, atherosclerosis or an ocular disease selected from the group consisting of diabetic retinopathy, exudative (wet) or nonexudative (dry) macular degeneration (AMD), corneal graft rejection, corneal neovascularisation, retinopathy of prematurity (ROP), retinal artery or vein occlusion, neovascular glaucoma and sickle cell retinopathy.

18. A method for the treatment of a disease associated with angiogenesis comprising administering to a subject in need thereof a therapeutically effective amount of a peptide according to any one of claims 1 to 3, a fusion polypeptide according to claims 4 to 7, a combination of peptides according to any one of claims 8 to 10, a nucleic acid construct according to claim 11 or a pharmaceutical composition according to claim 14.

19. A kit comprising a peptide according to any one of claims 1 to 3, a fusion polypeptide according to claims 4 to 7, a combination of peptides according to any one of claims 8 to 10, a nucleic acid construct according to claim 11 or a pharmaceutical composition according to claim 14.

20. A peptide for use in a method of treating a patient with a disease associated with vascular leakage, wherein the peptide comprises an amino acid sequence having at least 70% identity to SEQ ID NO: 3 or SEQ ID NO: 4, optionally wherein the peptide is as defined in any one of claims 8 to 10.

21. The peptide for use according to claim 20, wherein: (i) the peptide is administered as part of a combination according to claims 8 to 10; and/or (ii) said disease has not progressed to neovascularisation.

22. A peptide for use in a method of preventing progression of a disease associated with vascular leakage to a disease comprising neovascularisation, optionally wherein the peptide comprises an amino acid sequence having at least 70% identity to SEQ ID NO: 3 or SEQ ID NO: 4, optionally wherein the peptide is as defined in any one of claims 8 to 10.

23. The peptide for use according to any one of claims 20 to 22, wherein the disease is: (i) an ocular disease, optionally wherein the ocular disease is early-stage AMD, diabetic retinopathy or diabetic macular oedema; and/or (ii) associated with oedema and/or inflammation.

24. The peptide for use according to any one of claims 20 to 22, wherein the disease is selected from cancer, a solid tumour, rheumatoid arthritis, lymphoedema, asthma, ventilator induced-lung injury, acute lung injury, atherosclerosis, ischemic stroke, psoriasis, an inflammatory bowel disorder, and/or myocardial infarction, optionally wherein said cancer is ovarian cancer, or lung cancer, preferably epithelial ovarian cancer or nonsquamous non-small cell lung cancer, optionally wherein the disease is a solid tumour and the peptide is administered in combination with a chemotherapeutic agent or irradiation treatment, optionally thoracic irradiation treatment.

25. The peptide for use according to any one of claims 20 to 24, wherein the disease is refractory to treatment with an anti-VEGF antagonist.


SEQUENCE LISTING (27 sequences)

SEQ ID NO: 1 - protein, 9 aa, Homo sapiens
LPLPLTTVA

SEQ ID NO: 2 - protein, 27 aa, Homo sapiens
PPFTATTAVIRTTGVRRLLPLPLTTVA

SEQ ID NO: 3 - protein, 18 aa, Homo sapiens
PAEEDTNVYTEKHSDSLF

SEQ ID NO: 4 - protein, 18 aa, Mus musculus
PAIKSTDVYTEKHSDNLF

SEQ ID NO: 5 - protein, 201 aa, Homo sapiens
MRRAWILLTLGLVACVSAESRAELTSDKDMYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILLLVYRMRKKDEGSYDLGERKPSSAAYQKAPTKEFYA

SEQ ID NO: 6 - protein, 202 aa, Mus musculus
MQRAWILLTLGLMACVSAETRTELTSDKDMYLDNSSIEEASGVYPIDDDDYSSASGSGADEDIESPVLTTSQLIPRIPLTSAASPKVETMTLKTQSITPAQTESPEETDKEEVDISEAEEKLGPAIKSTDVYTEKHSDNLFKRTEVLAAVIAGGVIGFLFAIFLILLLVYRMRKKDEGSYDLGERKPSSAAYQKAPTKEFYA

SEQ ID NO: 7 - protein, 9 aa, Mus musculus
LPLPLTTAA

SEQ ID NO: 8 - protein, 442 aa, Homo sapiens
MKPGPPHRAGAAHGAGAGAGAAAGPGARGLLLPPLLLLLLAGRAAGAQRWRSENFERPVDLEGSGDDDSFPDDELDDLYSGSGSGYFEQESGIETAMRFSPDVALAVSTTPAVLPTTNIQPVGTPFEELPSERPTLEPATSPLVVTEVPEEPSQRATTVSTTMATTAATSTGDPTVATVPATVATATPSTPAAPPFTATTAVIRTTGVRRLLPLPLTTVATARATTPEAPSPPTTAAVLDTEAPTPRLVSTATSRPRALPRPATTQEPDIPERSTLPLGTTAPGPTEVAQTPTPETFLTTIRNEPEVPVSGGPSGDFELPEEETTQPDTANEVVAVGGAAAKASSPPGTLPKGARPGPGLLDNAIDSGSSAAQLPQKSILERKEVLVAVIVGGVVGALFAAFLVTLLIYRMKKKDEGSYTLEEPKQASVTYQKPDKQEEFYA

SEQ ID NO: 9 - protein, 442 aa, Mus musculus
MKPGPPRRGTAQGQRVDTATHAPGARGLLLPPLLLLLLAGRAAGAQRWRNENFERPVDLEGSGDDDSFPDDELDDLYSGSGSGYFEQESGLETAMRFIPDMALAAPTAPAMLPTTVIQPVDTPFEELLSEHPRPEPVTSPPLVTEVKEVVEESSQKATTISTTTSTTAATTTGAPTMATAPATAATTAPSTPEAPPATATVADVRTTGIQGMLPLPLTTAATAKITTPAAPSPPTTVATLDTEAPTPRLVNTATSRPQSLPRPITTQEPEVAERSTLPLGTTAPGPTEVAQTPTPESLLTTTQDEPEVPVSGGPSGDFELQEETTQPDTANEVVAVEGAAAKPSPPLGTLPKGARPGLGLHDNAIDSGSSAAQLLQKSILERKEVLVAVIVGGVVGALFAAFLVTLLIYRMKKKDEGSYTLEEPKQASVTYQKPDKQEEFYA

SEQ ID NO: 10 - DNA, 26 bp, Homo sapiens
  source 1..26: mol_type = genomic DNA
ctgcctctcccactgaccacagtggc

SEQ ID NO: 11 - DNA, 90 bp, Homo sapiens
  source 1..90: mol_type = genomic DNA
cctgcagcacccccttttacggccaccactgctgttataaggaccactggcgtacggaggcttctgcctctcccactgaccacagtggct

SEQ ID NO: 12 - DNA, 54 bp, Homo sapiens
  source 1..54: mol_type = genomic DNA
ccagccgaagaggatacaaatgtgtatactgagaaacactcagacagtctgttt

SEQ ID NO: 13 - DNA, 54 bp, Mus musculus
  source 1..54: mol_type = genomic DNA
cctgctataaaaagcacagatgtgtacacggagaaacattcagacaatctgttt

SEQ ID NO: 14 - DNA, 606 bp, Homo sapiens
  source 1..606: mol_type = genomic DNA
atgcggcgcgcgtggatcctgctcaccttgggcttggtggcctgcgtgtcggcggagtcgagagcagagctgacatctgataaagacatgtaccttgacaacagctccattgaagaagcttcaggagtgtatcctattgatgacgatgactacgcttctgcgtctggctcgggagctgatgaggatgtagagagtccagagctgacaacatctcgaccacttccaaagatactgttgactagtgctgctccaaaagtggaaaccacgacgctgaatatacagaacaagatacctgctcagacaaagtcacctgaagaaactgataaagagaaagttcacctctctgactcagaaaggaaaatggacccagccgaagaggatacaaatgtgtatactgagaaacactcagacagtctgtttaaacggacagaagtcctagcagctgtcattgctggtggagttattggctttctctttgcaatttttcttatcctgctgttggtgtatcgcatgagaaagaaggatgaaggaagctatgaccttggagaacgcaaaccatccagtgctgcttatcagaaggcacctactaaggagttttatgcgtaa

SEQ ID NO: 15 - DNA, 609 bp, Mus musculus
  source 1..609: mol_type = genomic DNA
atgcagcgcgcgtggatcctgctcaccttgggcttgatggcctgtgtgtccgcagagacgagaacagagctgacatccgataaggatatgtaccttgacaatagctccattgaggaagcttcaggagtatatcctattgatgatgatgactattcttctgcctcaggctcaggagctgatgaagacatagagagtccagttctgacaacatcccaactgattccaagaatcccactcactagtgctgcttcccccaaagtggaaaccatgacgttgaagacacaaagcattacacctgctcagactgagtcacctgaagaaactgacaaggaggaagttgacatttctgaggcagaagagaagctgggccctgctataaaaagcacagatgtgtacacggagaaacattcagacaatctgtttaaacggacagaagttctagcagccgtcattgctggtggtgtgatcggctttctctttgccattttcctcatcctgctattggtgtaccgcatgcggaagaaagatgaaggaagctacgaccttggagaacgcaaaccatccagcgcagcttaccagaaggcacccactaaggagttttatgcataa

SEQ ID NO: 16 - DNA, 83 bp, Mus musculus
  source 1..83: mol_type = genomic DNA
cgccccctgccacggctaccgtggctgacgtaaggaccaccggcatacaggggatgctgcctcttcccctgaccacagctgcc

SEQ ID NO: 17 - DNA, 1329 bp, Homo sapiens
  source 1..1329: mol_type = genomic DNA
atgaagccggggccgccgcaccgtgccggggccgcccacggggccggcgccggggccggggccgcggccgggcccggggcccgcgggctgctcctgccaccgctgctgctgctgctgctggcggggcgcgccgcgggggcccagcgctggcgcagtgagaacttcgagagacccgtggacctggagggctctggggatgatgactcctttcccgatgatgaactggatgacctctactcggggtcgggctcgggctacttcgagcaggagtcgggcattgagacagccatgcgcttcagcccagatgtagccctggcggtgtccaccacacctgcggtgctgcccaccacgaacatccagcctgtgggcacaccatttgaagagctcccctctgagcgccccaccctggagccagccaccagccccctggtggtgacagaagtcccggaagagcccagccagagagccaccaccgtctccactaccatggctaccactgctgccacaagcacaggggacccgactgtggccacagtgcctgccacagtggccaccgccacccccagcacccctgcagcacccccttttacggccaccactgctgttataaggaccactggcgtacggaggcttctgcctctcccactgaccacagtggctacggcacgggccactacccccgaggcgccctccccgcccaccacggcggctgtcttggacaccgaggccccaacacccaggctggtcagcacagctacctcccggccaagagcccttcccaggccggccaccacccaggagcctgacatccctgagaggagcaccctgcccctggggaccactgcccctggacccacagaggtggctcagaccccaactccagagaccttcctgaccacaatccgggatgagccagaggttccggtgagtggggggcccagtggagacttcgagctgccagaagaagagaccacacaaccagacacagccaatgaggtggtagctgtgggaggggctgcggccaaggcatcatctccacctgggacactgcccaagggtgcccgcccgggccctggcctcctggacaatgccatcgactcgggcagctcagctgctcagctgcctcagaagagtatcctggagcggaaggaggtgctcgtagctgtgattgtgggcggggtggtgggcgccctctttgctgccttcttggtcacactgctcatctatcgtatgaagaaaaaggatgagggcagctacacgctggaggaacccaagcaggcgagcgtcacataccagaagcctgacaagcaggaggagttctatgcctag

SEQ ID NO: 18 - DNA, 1329 bp, Mus musculus
  source 1..1329: mol_type = genomic DNA
atgaagcccgggccgccgcgccgcgggaccgcacaggggcagcgcgtggacaccgccacccatgcgcccggggcccgcgggctgttgctgccaccgctgctgctgctgctgctggccggccgcgccgcgggggctcaacgctggcgcaatgagaacttcgagaggccggtggatcttgagggctcaggggatgacgactcgtttcctgatgatgaactagacgacctctactcggggtcaggctctggctacttcgagcaggagtccggccttgagacggccatgcggttcatccctgatatggccctggctgcgcccactgcacctgccatgctacccacaaccgttatccagcccgtggacaccccatttgaggaactcctttctgagcaccccagccctgaaccagtcaccagtcccccgctggtgacagaggtgacagaggtcgtagaagagtccagccagaaagctaccaccatctctaccaccacatctaccaccgcggccaccaccacaggggccccaactatggccacagcacctgccacagcagccaccactgcccctagcactcccgaggcgccccctgccacggctaccgtggctgacgtaaggaccaccggcatacaggggatgctgcctcttcccctgaccacagctgccacagccaagatcactaccccagcagcaccctcaccacccactactgtggctaccttggacacagaggccccgacacctaggctggtcaacacagctacctcgaggccacgagcccttcctcggccagtcaccacccaggagcctgatgttgctgagaggagtaccctgccgttggggaccacggctcctggacccacggagatggctcagaccccaactccagagtcccttctgaccaccatccaggatgagccagaggtgccagtaagtggggggcccagcggggactttgagcttcaagaagagaccacgcagccggacacggccaatgaggtggtggctgtggaaggagccgcggccaagccgtcacctccactggggacactgcccaagggtgcccgcccaggccctggcctccacgacaatgccatcgattcgggcagctcggccgcccagctccctcagaagagcatactggagcggaaggaggtgctcgtagccgtgatcgtgggtggggtggtgggcgccctcttcgctgccttcctggtcacgctgctcatctaccgcatgaagaagaaggacgaaggcagctacaccttggaagaacccaagcaggcaagcgtcacgtaccagaaacctgacaagcaggaggagttctacgcttag

SEQ ID NO: 19 - protein, 27 aa, synthetic construct
LPLPLTTVAPAEEDTNVYTEKHSDSLF

SEQ ID NO: 20 - protein, 27 aa, synthetic construct
PAEEDTNVYTEKHSDSLFLPLPLTTVA

SEQ ID NO: 21 - protein, 30 aa, synthetic construct
LPLPLTTVAGGSPAEEDTNVYTEKHSDSLF

SEQ ID NO: 22 - protein, 30 aa, synthetic construct
PAEEDTNVYTEKHSDSLFGGSLPLPLTTVA

SEQ ID NO: 23 - protein, 18 aa, Mus musculus
PPATATVADVRTTGIQGL

SEQ ID NO: 24 - protein, 8 aa, Mus musculus
PLPLTTAA

SEQ ID NO: 25 - protein, 27 aa, Mus musculus
PPATATVADVRTTGIQGMLPLPLTTAA

SEQ ID NO: 26 - protein, 24 aa, Homo sapiens
TATTAVIRTTGVRRLLPLPLTTVA

SEQ ID NO: 27 - protein, 17 aa, Homo sapiens
RTTGVRRLLPLPLTTVA